(19) United States
(12) Patent Application Publication
(10) Pub. No.: US 2005/0065195 A1
(43) Pub. Date: Mar. 24, 2005

(54) Oxadiazolyl-biphenylcarboxamides and their use as p38 kinase inhibitors

(76) Inventors: Richard Martyn Angell, London (GB); Paul Bamborough, Stevenage (GB); George Stuart Cockerill, London (GB); Kathryn Jane Smith, Stevenage (GB); Ann Louise Walker, Stevenage (GB)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/492,711

(22) PCT Filed: Oct. 16, 2002
(86) PCT No.: PCT/EP02/11574

(30) Foreign Application Priority Data
Oct. 17, 2001 (GB) 01249325

(51) Int. Cl. A61K 31/4245; A61K 31/4172; A61K 31/277; A61K 31/195

(52) U.S. Cl. 514/364; 514/400; 514/524; 514/567; 514/602; 514/649; 514/640; 564/86; 564/162; 564/163; 564/253; 564/338

(57) ABSTRACT

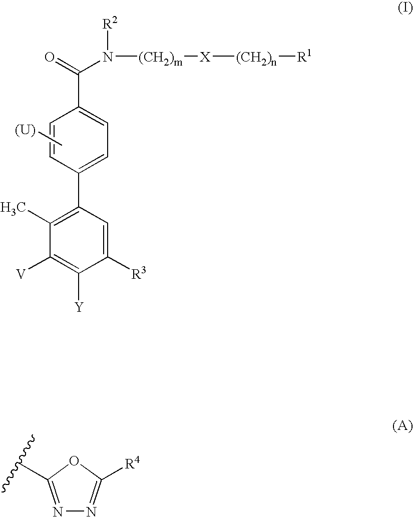

Compounds of formula (I), wherein R3 is the group; or pharmaceutically acceptable salts or solvates thereof, and their use as pharmaceuticals, particularly as p38 kinase inhibitors.

This invention relates to novel aniline and benzanilide derivatives, to processes for their preparation, and to pharmaceutical compositions containing them.

According to the present invention there is provided compounds of the general formula (I): 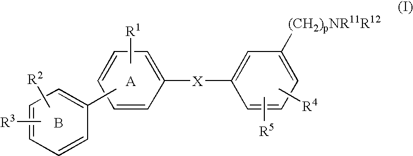
or a physiologically acceptable salt or solvate thereof, in which
R¹ represents a hydrogen atom or a halogen atom or a C₁₋₆alkyl or C₁₋₆alkoxy group;
R² and R³, which may be the same or different, each independently represent a hydrogen atom or a halogen atom, or C₁₋₆alkyl, hydroxyC₁₋₆alkyl, C₁₋₆alkoxy, hydroxy, —CF₃, —CN, —NO₂, —CO₂R¹⁰, —COR⁶, —SR⁶, —SOR⁶, —SO₂R⁶, —CR⁶═NOR⁷, —CONR⁶R⁷, —CONR⁶SO₂R⁷, —CONR⁶(CH₂)ₘCO₂R⁷, —CONR⁶(CH₂)ₘOC₁₋₄alkyl, —SO₂NR⁶R⁷, —OC(O)NR⁶R⁷, —(CH₂)ₙNR⁸R⁹, —(CH₂)ₙOC(O)C₁₋₄alkyl (optionally substituted by a C₁₋₆alkoxy group), or C₁₋₄alkoxyalkyl (optionally substituted by a C₁₋₄alkoxy or hydroxy group);
or R² represents a 1,2,4-oxadiazol-3-yl, 1,2,4-oxadiazol-5-yl, 1,3,4-oxadiazol-2-yl, 1,2,5-oxadiazol-3-yl, imidazol-1-ylmethyl, dioxolan or thioxolan group, each of which may be optionally substituted by a C₁₋₃alkyl group;
or, when R² and R³ are attached to adjacent carbon atoms, they may form a 5- or 6-membered saturated fused ring which contains one or two oxygen atoms and which may be optionally substituted by an oxo group;
R⁴ and R⁵, which may be the same or different, each independently represent a hydrogen atom or a halogen atom, or a hydroxy, C₁₋₆alkoxy or C₁₋₆alkyl group;
R⁶, R⁷ and R⁸ which may be the same or different, each independently represent a hydrogen atom or a C₁₋₆alkyl group;
or —NR⁶R⁷ forms a saturated heterocyclic ring which has 5 or 6 ring members which, when there are 6 ring members, may optionally contain in the ring one oxygen or sulphur atom;
R⁹ represents a hydrogen atom or a C₁₋₆alkyl, —COR¹³ or —SO₂R¹⁴ group;
or —NR⁸R⁹ forms a saturated heterocyclic ring which has 5 or 6 ring members, may optionally be substituted by an oxo group and, when there are 6 ring members, may optionally contain in the ring one oxygen or sulphur atom;
R¹⁰ represents a hydrogen atom or a C₁₋₆alkyl group optionally substituted by one or two substituents selected from C₁₋₆alkyl, C₁₋₆alkoxy, hydroxy or —NR⁶R⁷;
R¹¹ and R¹² which may be the same or different, each independently represent a hydrogen atom or a C₁₋₆alkyl group;
R¹³ represents a hydrogen atom or a group selected from C₁₋₆alkyl, C₁₋₆alkoxy or a C₁₋₄alkoxyalkyl group;
R¹⁴ represents a C₁₋₆alkyl or phenyl group,
X represents —CONH—, —NHCO—, —CH₂NH— or —NHCH₂—;
m represents an integer from 1 to 3;
n represents zero or an integer from 1 to 3; and
p represents an integer from 2 to 4.

It is to be understood that the present invention encompasses all geometric and optical isomers of the compounds of general formula (I) and their mixtres including the racemic mitres thereof.

Physiologically acceptable salts of the compounds of formula (I) include acid addition salts formed with inorganic or organic acids (for example hydrochlorides, hydrobromides, sulphates, phosphates, benzoates, naphthoates, hydroxynaphthoates, p-toluenesulphonates, methanesulphonates, sulphamates, ascorbates, tartrates, citrates, oxalates, maleates, salicylates, fumarates, succinates, lactates, glutarates, glutaconates, acetates or tricarballylates) and, where appropriate, inorganic base salts such as alkali metal salts (for example sodium salts).

In the compounds of formula (I), the term “C₁₋₆alkyl” or “C₁₋₆alkoxy” as a group or part of a group means that the group is straight or branched and consists of 1 to 6 carbon atoms. Examples of suitable alkyl groups include methyl, ethyl, n-propyl, i-propyl, n-butyl, s-butyl and t-butyl. Examples of suitable alkoxy groups include methoxy, ethoxy, n-propoxy, i-propoxy, n-butoxy, s-butoxy and t-butoxy. The term “halogen” within the definition of R² means fluorine, chlorine, bromine or iodine.

Within the above definition, when R² and R³ are attached to adjacent carbon atoms and together form a 5- or 6-membered saturated fused ring which contains one or two oxygen atoms and which may be optionally substituted by an oxo group, suitable groups represented by —R²—R³— include —C(O)OCH₂—, —OCH₂O— or —CH₂OCH₂O—.

Within the above definition, when —NR⁶R⁷ or —NR⁸R⁹ represent a saturated heterocyclic ring, these contain 5 or 6 ring members, one of which (when there are 6 ring members) may be an oxygen or a sulphur atom. Suitable heterocyclic groups are a pyrrolidinyl, piperidinyl, morpholinyl or thiomorpholinyl group.

Where a saturated heterocyclic ring is formed by the group —NR⁸R⁹ and said ring is substituted by an oxo group, suitable heterocyclic groups include a 2-oxo-1-pyrrolidinyl, 4-oxo-3-thiazolidinyl or 2-oxo-tetrahydro-1,3-thiaziryl group.

The phenyl ring B may preferably be attached in the meta or more particularly the para position of the phenyl ring A relative to the group X.

When the phenyl ring B is substituted by a single atom or group as defined above the substituent is preferably attached in a position meta or para to the phenyl ring A in general formula (I). When the phenyl ring B is substituted by two atoms or groups as defined above one substituent is preferably attached in the position para to, and the other is in a position ortho to the phenyl ring A in general formula (I).

A preferred group of compounds of general formula (I) is that wherein the phenyl ring B is substituted by one or two substituents as defined in general formula (I) wherein one substituent is in the position para to the phenyl ring A in general formula (I) the second substituent is in the position ortho to the phenyl ring A in general formula (I).

Another preferred group of compounds of general formula (I) is that wherein the phenyl ring B is substituted by a single substituent as defined in general formula (I) wherein said substituent is in the position para to the phenyl ring A in general formula (I).

A further preferred group of compounds of general formula (I) is that wherein R² and R³ each independently represent a hydrogen atom or a halogen atom; or a C₁₋₆alkyl, especially methyl, group; hydroxymethyl; hydroxy; —CN; —CO₂R¹⁰ where R¹⁰ is a C₁₋₆alkyl, especially methyl or ethyl, group optionally substituted by a C₁₋₆alkoxy, especially methoxy, group; —COR⁶ where R⁶ is a C₁₋₆alkyl, especially methyl, ethyl, propyl or butyl, group; —SR⁶ where R⁶ is a C₁₋₆alkyl, especially methyl, group; —SOR⁶ where R⁶ is a C₁₋₆alkyl especially methyl, group; —CR⁶═NOR⁷ where R⁶ is a hydrogen atom or a C₁₋₆alkyl especially methyl group, and R⁷ is a hydrogen atom or a C₁₋₆alkyl, especially methyl, group; —CONR⁶R⁷ where R⁶ and R⁷ each independently represent C₁₋₆alkyl, especially methyl, groups or —NR⁶R⁷ forms a saturated heterocyclic group which has six members and contains in the ring one oxygen atom, especially a morpholinyl ring; —CONR⁶(CH₂)ₘOC₁₋₄alkyl, where R⁶ is a C₁₋₆alkyl especially methyl, group and m is two, especially the group —CON(CH₃)(CH₂)₂OCH₃; —SO₂NR⁶R⁷ where R⁶ and R⁷ each independently represent a hydrogen atom or a C₁₋₆alkyl, especially methyl, group; —OC(O)NR⁶R⁷ where R⁶ and R⁷ each independently represent a C₁₋₆alkyl, especially methyl group; —(CH₂)ₙNR⁸R⁹ where R⁸ is a hydrogen atom or a C₁₋₆alkyl, especially methyl, group, R⁹ is a C₁₋₆alkyl, especially methyl, group, or —COR¹³ (where R¹³ is a C₁₋₆alkyl, especially methyl group, a C₁₋₆alkoxy, especially methoxy or ethoxy, group or a C₁₋₄alkoxyalkyl, especially methoxymethyl, group) or —SO₂R¹⁴ (where R¹⁴ is a C₁₋₆alkyl, especially methyl, group), or —NR⁸R⁹ forms a saturated heterocyclic group which has six ring members and contains in the ring one oxygen or sulphur atom, especially a morpholinyl, thiomorpholinyl or 2-oxo-1-pyrrolidinyl ring, and n is zero, 1 or 2; or a C₁₋₄alkoxyalkyl, especially methoxymethyl or methoxyethyl, group substituted by a C₁₋₄alkoxy, especially methoxy, group.

Another preferred group of compounds of general formula (I) is that wherein R² represents a hydrogen atom, a chlorine atom or a methyl group and R³ represents hydroxymethyl, hydroxy, —CO₂CH₂CH₂OCH₃, —COCH₃, —SOCH₃, —C(CH₃)═NOH, —CON(CH₃)₂, —SO₂NH₂, —SO₂NHCH₃, —SO₂N(CH₃)₂, —OC(O)N(CH₃)₂, —NHCH₃, —N(CH₃)₂, —N(CH₃)COCH₃, —CH₂NHCO₂CH₂CH₃, —CH₂N(CH₃)COCH₂OCH₃, —NHSO₂CH₃, 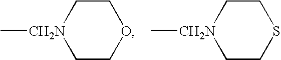
or CH₂OCH₂CH₂OCH₃.

Also preferred is the group of compounds of general formula (I) wherein R¹ is a hydrogen atom or a C₁₋₄alkyl, especially methyl, group.

Another preferred group of compounds of general formula (I) is that wherein R¹ is attached at a position ortho to the phenyl ring B on the phenyl ring A in general formula (I).

Another preferred group of compounds of general formula (I) is that wherein R⁴ is attached in the para-position relative to the group X.

A further preferred group of compounds of general formula (I) is that wherein R⁴ is a halogen atom, especially a fluorine or chlorine atom, or a hydroxy or C₁₋₆alkoxy, especially methoxy, group.

Also preferred is the group of compounds of general formula (I) wherein R⁵ is a hydrogen atom or a fluorine atom.

A yet further preferred group of compounds of general formula (I) is that wherein R¹¹ and R¹² each represent a C₁₋₆alkyl, especially methyl, group.

Also preferred is the group of compounds of general formula (I) in which X is —NHCO— or —CONH—.

Another preferred group of compounds of general formula (1) is that wherein p is 3.

Preferred compounds of general formula (I) include:
4′-[[3-[3-(dimethylamino)propyl]-4-methoxybenzoyl]amino][1,1′-biphenyl]-4-carboxylic acid;
3-[3-(dimethylamino)propyl]-N-[4′-(1-hydroxyethyl)[1,1′-biphenyl]-4-yl]-4-methoxybenzamide;
N-[4′-(aminosulphonyl)[1,1′-biphenyl]-4-yl]-3-[3-(dimethylamino)propyl]-4-methoxybenzamide;
3-[3-(dimethylamino)propyl]-N-[4′-(hydroxymethyl)[1,1′-biphenyl]-4-yl]-4-methoxybenzamide;
N-[[4′-[[3-[3-(dimethylamino)propyl]-4-methoxybenzoyl]amino][1,1′-biphenyl]-4-yl]carbonyl]glycine;
3-[3-(dimethylamino)propyl]-N-[4′-(hydroxymethyl-2,2′-dimethyl[1,1′-biphenyl]-4-yl]-4-methoxybenzamide;
3-[3-(dimethylamino)propyl]-N-[4′-(hydroxymethyl)-2′-methyl[1,1′-biphenyl]-4-yl]-4-methoxybenzamide;
4′-[[3-[3-(dimethylamino)propyl]-4-hydroxybenzoyl]amino][1,1′-biphenyl]-4-carboxamide;
3-[3-(dimethylamino)propyl]-N-(4′-hydroxy-2-methyl[1,1′-biphenyl]-4-yl)-4-methoxybenzamide;
N-[4-(3-oxa-1H-isobenzofuran-6-yl)phenyl]-3-[3(dimethylamino)propyl]-4-methoxybenzamide;
3-[3-(dimethylamino)propyl]-N-[4′-(1H-imidazol-1-ylmethyl)[1,1′-biphenyl]4-yl]4-methoxybenzamide;
and their physiologically acceptable salts and solvates.

Particularly preferred compounds of general formula (I) include:
N-(4′-acetyl[1,1′-biphenyl]-4-yl)-3-[3-(dimethylamino)propyl]-4-methoxybenzamide;
4′-[[3-[3-(dimethylamino)propyl]-4-methoxybenzoyl]amino][1,1′-biphenyl]-4-carboxamide;
4′-[[3-[3-(dimethylamino)propyl]-4-methoxybenzoyl]amino]-3-hydroxy[1,1′-biphenyl]-4-carboxylic acid;
3-[3-(dimethylamino)propyl]-N-(4′-hydroxy[1,1′-biphenyl]4-yl)-4-methoxybenzamide;
4′-[[3-[3-(dimethylamino)propyl]-4-methoxybenzoyl]amino]-2-methyl[1,1′-biphenyl]-4-carboxylic acid;
N-(2′-chloro-4′-formyl[1,1′-biphenyl]-4-yl)-3-[3-(dimethylamino)propyl]-4-methoxybenzamide;
3-[3-(dimethylamino)propyl]-N-(4′-formyl-2′-methyl[1,1′-biphenyl)-4-yl)-4-hydroxybenzamide;
N-(4′-acetyl-2′-methyl[1,1′-biphenyl]-4-yl)-3-[3-(dimethylamino)propyl]-4-methoxybenzamide;
3-[3-(dimethylamino)propyl]-N-[4′-formyl[1,1′-biphenyl]-4-yl)-4-methoxybenzamide;
(±)-[4′-[[3-[3-(dimethylamino)propyl]-4-methoxybenzoyl]amino]-3-methyl[1,1′-biphenyl]-4-yl]methyl 2-methoxypropanoate;
and their physiologically acceptable salts and solvates.

5-Hydroxytryptamine (serotonin) is a neurotransmitter which is widely distributed within the central nervous system (CNS), platelets and the gastrointestinal tract. Changes in transmission in serotonergic pathways in the CNS are known to modify, for example, mood, psychomotor activity, appetite, memory and blood pressure. Release of 5-hydroxytryptamine from platelets can mediate vasospasm while changes in free 5-hydroxytryptamine levels in the gastrointestinal tract can modify secretion and motility.

Abundant pharmacological studies have led to the discovery of multiple types of receptors for 5-hydroxytryptamine, thus providing a molecular basis to the diversity of its actions. These receptors are classed as 5-HT₁, 5-HT₂ and 5-HT₃, with 5-HT₁ receptors being sub-classified as 5-HT_{1A}, 5-HT_{1B}, 5-HT_{1C}, 5-HT_{1D} and 5-HT_{1D}(like) receptors. The identification of these classes and subclasses of receptor is based mainly on radioligand binding studies.

Compounds having a selective antagonist action at 5-HT_{1D} receptors such as those described herein may exhibit a beneficial effect on subjects suffering from CNS disorders.

In the present specification, a 5-HT_{1D} antagonist is a non-naturally occurring (synthetic) compound that specifically and selectively antagonises 5-HT_{1D} receptors, i.e. blocks the specific actions of 5-hydroxytryptamine mediated by 5-HT_{1D} receptors. Such compounds may be identified by a high level of affinity (pKi≧8) in the in vitro human cortex and guinea-pig striatum radioligand binding assays described by Hoyer et al, Neuroscience Letters, 1988, 85, p357-362. Activity at 5-HT_{1D} receptors may be confirmed in vivo using the guinea pig rotation model described by G A Higgins et al, Br. J. Pharmacol., 1991, 102, p305-310.

The affinity of a compound for 5-HT_{1A}, 5-HT_{1C} and/or 5-HT₂ receptors is measured using the in vitro tests described in the following publications:
5-HT_{1A} Gozian et al, Nature, 1983, 305, p140-142
5-HT_{1C} Pazos et al, Eur. J.Pharmacol., 1984, 106, p531-538
5-HT₂ Humphrey et al, Br. J. Pharmacol, 1988, 94 p1123-1132 (rabbit aorta model).

Thus, for example, compounds of the present invention have been shown to inhibit 5-hydroxytryptamine induced contraction of the dog isolated saphenous vein and to antagonise the 5-hydroxytryptamine induced inhibition of neurotransmission in central and peripheral neurones.

5-HT_{1D} antagonists, and in particular the compounds of the present invention, may therefore be of use in the treatment of CNS disorders such as mood disorders, including depression, seasonal affective disorder and dysthymia; anxiety disorders, including generalised anxiety, panic disorder, agoraphobia, social phobia, obsessive compulsive disorder and post-traumatic stress disorder, memory disorders, including dementia, amnestic disorders and age-associated memory impairment; and disorders of eating behaviour, including anorexia nervosa and bulimia nervosa. Other CNS disorders include Parkinson's disease, dementia in Parkinson's disease, neuroleptic-induced parkinsonism and tardive dyskinesias, as well as other psychiatric disorders.

5-HT_{1D} antagonists, and in particular compounds of the present invention, may also be of use in the treatment of endocrine disorders such as hyperprolactinaemia, the treatment of vasospasm (particularly in the cerebral vasculature) and hypertension, as well as disorders in the gastrointestinal tract where changes in motility and secretion are involved. They may also be of use in the treatment of sexual dysfunction. Therefore, according to a second aspect of the invention, we provide a compound of general formula (I) or a physiologically acceptable salt or solvate thereof for use in therapy.

According to a further aspect of the present invention, we therefore provide a compound of general formula (I) or a physiologically acceptable salt or solvate thereof for use in the treatment of the aforementioned disorders.

According to another aspect of the invention, we provide the use of a compound of general formula (I) or a physiologically acceptable salt or solvate thereof for the manufacture of a therapeutic agent for the treatment of the aforementioned disorders.

According to a further aspect of the invention, we provide, a method of treating the aforementioned disorders which comprises administering an effective amount to a patient in need of such treatment of a compound of general formula (I) or a physiologically acceptable salt or solvate thereof.

In particular, according to another aspect of the present invention, we provide a compound of general formula (I) or a physiologically acceptable salt or solvate thereof for use in the treatment or prophylaxis of depression.

It will be appreciated that the compounds according to the invention may advantageously be used in conjunction with one or more other therapeutic agents, for instance, different antidepressant agents such as tricyclic antidepressants (e.g. amitriptyline, dothiepin, doxepin, trimipramine, butriptyline, clomipramine, desipramine, imipramine, iprindole, lofepramine, nortriptyline or protriptyline), monoamine oxidase inhibitors (e.g. isocarboxazid, pheneizine or tranylcyclopramine) or 5-HT reuptake inhibitors (e.g. fluvoxamine, sertraline, fluoxetine or paroxetine), and/or antiparkinsonian agents such as dopaminergic antiparkinsonian agents (e.g. levodopa, preferably in combination with a peripheral decarboxylase inhibitor e.g. benserazide or carbidopa), or a dopamine agonist (e.g. bromocriptine, lysuride or pergolide). It is to be understood that the present invention covers the use of a compound of general formula (I) or a physiologically acceptable salt or solvate thereof in combination with one or more other therapeutic agents.

Thus there is provided in a further or alternative aspect of the present invention a compound of general formula (1) or a physiologically acceptable salt or solvate thereof and an antidepressant agent in the presence of each other in the human or non-human animal body for use in the treatment of the aforementioned disorders.

While it is possible that a compound of general formula (I) may be administered as the raw chemical it is preferable to present the active ingredient as a pharmaceutical formulation.

The compounds of general formula (I) and their physiologically acceptable salts and solvates may be formulated for administration in any convenient way, and the invention therefore also includes within its scope pharmaceutical compositions comprising at least one compound of general formula (I) or a physiologically acceptable salt or solvate thereof Such compositions may be presented for use in a conventional manner in admixture with one or more physiologically acceptable carriers or excipients.

The carrier(s) must be “acceptable” in the sense of being compatible with the other ingredients of the formulation and not deleterious to the recipient thereof.

Thus, the compositions according to the invention may be formulated for oral, buccal, parenteral or rectal administration or in a form suitable for administration by inhalation or insufflation. Oral administration is preferred.

Tablets and capsules for oral administration may contain conventional excipients such as binding agents, for example, syrup, acacia, gelatin, sorbitol, tragacanth, mucilage of starch or polyvinylpyrrolidone; fillers, for example, lactose, sugar, microcrystalline cellulose maize-starch, calcium phosphate or sorbitol; lubricants, for example, magnesium stearate, stearic acid, talc, polyethylene glycol or silica; disintegrants, for example, potato starch or sodium starch glycollate; or wetting agents such as sodium lauryl sulphate. The tablets may be coated according to methods well known in the art. Oral liquid preparations may be in the form of for example, aqueous or oily suspensions, solutions, emulsions, syrups or elixirs, or may be presented as a dry product for constitution with water or other suitable vehicle before use. Such liquid preparations may contain conventional additives such as suspending agents, for example, sorbitol syrup, methylcellulose, glucose/sugar syrup, gelatin, hydroxypropyl methylcellulose, carboxymethylcellulose, aluminium stearate gel or hydrogenated edible fats; emulsifying agents, for example, lecithin, sorbitan mono-oleate or acacia; non-aqueous vehicles (which may include edible oils), for example, almond oil, fractionated coconut oil, oily esters, propylene glycol or ethyl alcohol; and preservatives, for example, methyl or propyl p-hydroxybenzoates or sorbic acid. The compositions may also be formulated as suppositories, e.g. containing conventional suppository bases such as cocoa butter or other glycerides.

For buccal administration the composition may take the form of tablets or lozenges formulated in conventional manner.

The composition according to the invention may be formulated for parenteral administration by bolus injection or continuous infusion. Formulations for injection may be presented in unit dose form in ampoules, or in multi-dose containers with an added preservative. The compositions may take such forms as suspensions, solutions, or emulsions in oily or aqueous vehicles, and may contain formulatory agents such as suspending, stabilising and/or dispersing agents. Alternatively the active ingredient may be in powder form for constitution with a suitable vehicle, e.g. sterile, pyrogen-free water, before use.

For administration by inhalation either orally or nasally the compositions according to the invention are conveniently delivered in the form of an aerosol spray presentation from pressurised packs with the use of a suitable propellant, e.g. dichlorodifluoromethane, trichlorofluoromethane, dichlorotetrafluoroethane, carbon dioxide or other suitable gas, or from a nebuliser. In the case of a pressurised aerosol the dosage unit may be determined by providing a valve to deliver a metered amount.

Alternatively, for administration by inhalation the compositions according to the invention may take the form of a dry powder composition, for example a powder mix of the compound and a suitable powder base such as lactose or starch. The powder composition may be presented in unit dosage form in, for example, capsules or cartridges of e.g. gelatin, or blister packs from which the powder may be administered with the aid of an inhaler or insufflator.

The pharmaceutical formulations according to the invention may also contain other active ingredients such as antimicrobial agents, or preservatives.

The compositions according to the invention may be prepared by mixing the various ingredients using conventional means.

It will be appreciated that the amount of a compound of general formula (I) required for use in treatment will vary not only with the particular compound selected but also with the route of administration, the nature of the condition being treated and the age and condition of the patient and will ultimately be at the discretion of the attendant physician or veterinarian. In general, however, a proposed dose of the compounds of the invention for administration in man is 0.5 to 1000 mg, preferably 1 to 200 mg of the active ingredient per unit dose which could be administered, for example, 1 to 4 times per day.

The compounds of the invention may be prepared by a number of processes as described in the following. In describing the processes which may be used for preparing the compounds of general formula (I) or intermediates useful in the preparation thereof, any of R¹—R¹⁴, m and n in the various formulae are as defined in general formula (I) unless otherwise stated.

It will be appreciated that in the following methods for the preparation of compounds of general formula (I), for certain reaction steps it may be necessary to protect various reactive substituents in the starting materials for a particular reaction and subsequently to remove the protecting group. Such protection and subsequent deprotection may be particularly pertinent where R⁶, R⁷, R⁸, R⁹, R¹⁰, R¹¹ and/or R¹² in intermediates used to prepare compounds of general formula (I) are hydrogen atoms. Standard protection and deprotection procedures can be employed, for example formation of a phthalimide (in the case of a primary amine), benzyl, trityl, benzyloxycarbonyl or trichloroethoxycarbonyl derivatives. Subsequent removal of the protecting group is achieved by conventional procedures. Thus a phthalimide group may be removed by treatment with hydrazine or a primary amine, for example methylamine. Benzyl or benzyloxycarbonyl groups may be removed by hydrogenolysis in the presence of a catalyst e.g. palladium, and trichloroethoxycarbonyl derivatives may be removed by treatment with zinc dust. Trityl groups may be removed under acidic conditions using standard procedures. It may also be necessary in some cases to protect carboxylic acid groups (e.g. as esters) or aldehyde or ketone groups (e.g. as acyclic or cyclic acetals or ketals or as thioacetals or thioketals). Subsequent removal of these protecting groups is achieved by conventional procedures. Thus for example alkyl esters may be removed under conditions of acidic or basic hydrolysis, benzyl esters may be removed by hydrogenolysis in the presence of a catalyst e.g. palladium. Acyclic or cyclic acetals or ketals may be removed under conditions of acidic hydrolysis and thioacetals and thioketals may be removed using a mercuric salt.

Hydroxyl groups may also need protection and these may be adequately protected under amenable conditions as their esters or tralkylsilyl, tetrahydropyran and benzyl ethers. Such derivatives may be deprotected by standard procedures.

According to one general process (1A), the compounds of general formula (I) in which X represents the group —CONH—, may be prepared by a carbonylation reaction involving an aniline (II) 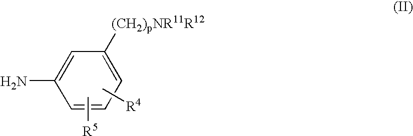
(where R⁴, R⁵, R¹¹, R¹² and p are as defined in general formula (I)) and a halophenyl compound (III) 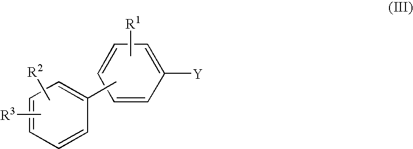
(where R¹, R² and R³ are as defined in general formula (I) and Y is a bromine or iodine atom or the group —OSO₂CF₃).

Alternatively, according to the general process (1B), the compounds of general formula (I), in which X represents the group —NHCO—, may be prepared by a carbonylation reaction involving a halophenyl compound (IV) 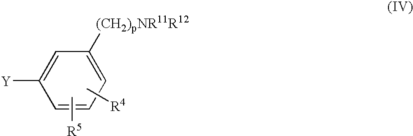
(where R⁴, R⁵, R¹¹, R¹² and p are as defined in general formula (I) and Y represents a bromine or iodine atom or the group —OSO₂CF₃) and an aniline of formula (V) 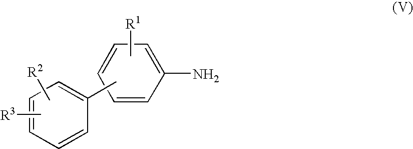
(where R¹, R² and R³ are as defined in general formula (I)).

Both reactions take place, for example, in the presence of carbon monoxide using a palladium salt as a catalyst. The reaction is effected in the presence of a suitable base e.g. a trialkylamine such as triethylamine or tri-n-butylamine and may be conducted in a suitable solvent such as an amide e.g. dimethylformamide or a nitrile e.g. acetonitrile at a temperature within the range of −10° C. to +150° C.

Suitable palladium salts for the reaction include triarylphosphine palladium (II) salts such as bis(triphenylphosphine)palladium (II) chloride.

According to another general process (2A), the compounds of general formula (I), in which X represents the group —CONH—, may be prepared by reacting an aniline of formula (II) with an activated carboxylic acid derivative of formula (VI) 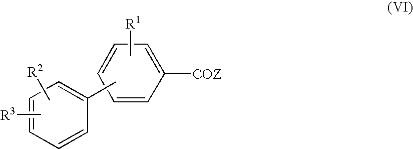
(where Z is a leaving group).

Alternatively, according to the general process (2B), the compounds of general formula (I), in which X represents the group —NHCO—, may be prepared by reacting an aniline of formula (V) with an activated carboxylic acid derivative of formula (VII) 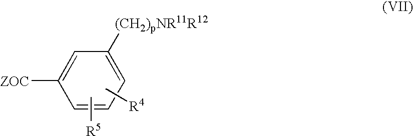
(where Z is a leaving group).

Suitable activated carboxylic acid derivatives represented in formulae (VI) and (VII) include acyl halides (e.g. acid chlorides) and acid anhydrides including mixed anhydrides. These activated derivatives may be formed from the corresponding acids of formulae (VIII) or (IX) 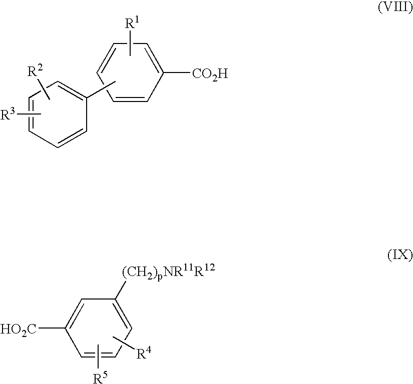
respectively, by well known procedures. For example, acid chlorides may be prepared by reaction with phosphorus pentachloride, thionyl chloride or oxalyl chloride and acid anhydrides may be prepared by reaction with an appropriate acid anhydride (e.g. trifluoroacetic anhydride), an acid chloride (e.g. acetyl chloride), an alkyl or aralkyl haloformate (e.g. ethyl or benzyl chloroformate) or methanesulphonyl chloride.

Activated carboxylic acid derivatives of formulae (VI) and (VII) may also be prepared in situ by the reaction of the corresponding acids of formulae (VIII) and (IX), respectively, with a coupling reagent such as 1,1′-carbonyldiimidazole, dicyclohexylcarbodiimide or diphenylphosphorylazide.

The conditions under which the activated carboxylic acid derivatives of formulae (VI) and (VII) are formed and subsequently reacted with the anilines of formulae (II) and (V), respectively, will depend upon the nature of the activated derivative. However, in general the reaction between the compounds (II) and (VI), or (V) and (VII), may be carried out in a non-aqueous medium such as, for example, dimethylformamide, tetrahydrofuran, acetonitrile of a halohydrocarbon such as dichloromethane at a temperature within the range −25° C. to +150° C. The reaction may optionally be carried out in the presence of a base such as triethylamine or pyridine and the base may also be used as the solvent for reaction.

Where acid chlorides are used, the reaction may be carried out using the Schotten-Baumann technique in the presence of a suitable base, for example, aqueous sodium hydroxide, conveniently at a temperature between 0° C. and 100° C., for example, room temperature.

According to another general process (3A), the compounds of general formula (I) may be prepared by treating a compound of formula (Xa) 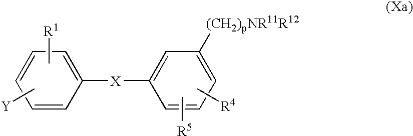
(where Y represents a bromine or iodine atom or the group —OSO₂CF₃) with a compound of formula (XIa) 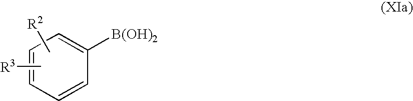
or an ester, an anhydride or a salt (e.g. lithium) thereof.
Alternatively, according to the general process (3B), the compounds of general formula (I) may be prepared by treating a compound of formula (Xb) 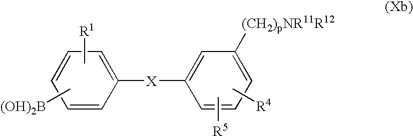
   or an ester, an anhydride or a salt (e.g. lithium) thereof, with a compound of formula (XIb) 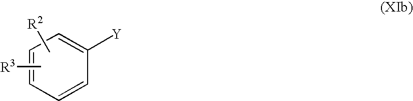
   where Y represents a bromine or iodine atom or the group —OSO₂CF₃.

Both reactions may be effected in the presence of a transition metal catalyst such as (Ph₃P)₄Pd (where Ph represents phenyl) in a suitable solvent such as an ether (e.g. 1,2-dimethoxyethane or tetrahydrofuran) in the presence or absence of water, or an aromatic hydrocarbon (e.g. benzene). The reaction is preferably carried out in the presence of a base such as an alkali or alkaline earth metal carbonate (e.g. sodium carbonate) at a suitable temperature up to reflux.

According to another general process (4), the compounds of general formula (I) may be prepared by reducing a compound of formula (XII) 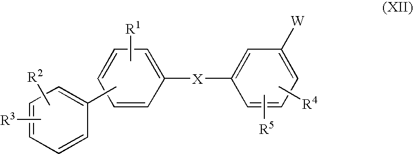
(where W represents a group convertible to the group —(CH₂)ₚNR¹¹R¹² under reducing conditions).

Examples of the type of group W which may be converted into the group —(CH₂)ₚNR¹¹R¹² are: —(CH₂)ₚ₋₁CN, —(CH₂)ₚ₋₁CHO, and when p is 3, —C≡CCN, —CH═CHCN, —CH═CHCHO, —CH═CHC2NR¹¹R¹² or —C≡CCHN₂R¹¹R¹². When W contains an aldehyde as defined above, the conversion is carried out in the presence of an appropriate amine of formula NHR¹¹R¹². When W contains a nitrile as defined above, the conversion may be carried out in the presence of an amine of formula NHR¹¹R¹², with the proviso that R¹¹ and R¹² do not both represent a hydrogen atom, in order to obtain a secondary or tertiary amine of general formula (I).

The reaction may be effected using an alkali or alkaline earth metal borohydride, e.g. sodium borohydride, or hydrogen and a metal catalyst such as palladium or platinum or oxides thereof The reaction may be carried out at a temperature between 0° C. and 100° C., conveniently at room temperature, and preferably in a solvent.

Suitable solvents for chemical reduction include ethers e.g. tetrahydrofuran, or alcohols e.g. ethanol. Suitable solvents for catalytic reduction include alcohols e.g. ethanol, ethers e.g. dioxan, amides e.g. dimethylformamide or a mixture of solvents e.g. ethanol/dimethylformamide.

According to another general process (5), the compounds of general formula (I) in which X represents either of the groups —NHCH₂— or —CH₂NH— may be prepared by reduction of the corresponding compounds of general formula (I) in which X represents the groups —NHCO— or —CONH—, respectively, except that the reaction cannot be used to prepare compounds in which R² and/or R³ represents another group reducible under the reaction conditions, for example, CONR⁶R⁷, CONR⁶(CH₂)ₘCO₂R⁷, SO₂R⁶, CO₂H, COR⁶, SOR⁶, CN, NO₂ or —(CH₂)ₙOC(O)C₁₋₄alkyl.

The reduction may be effected using a suitable metal hydride such as lithium aluminium hydride in a solvent e.g. an ether (such as tetrahydrofuran) at a temperature in the range of −10° C. to +100° C.

According to another general process (6A), the compounds of general formula (I) in which X represents the group —NHCH₂13 may be prepared by reacting an aniline of formula (V) with an aldehyde of formula (XXIII) 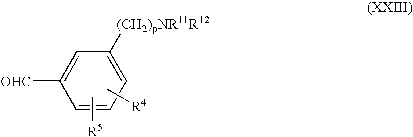
under reducing conditions.

Alternatively, according to general process (6B), the compounds of general formula (I) in which X represents the group —CH₂NH— may be prepared by reacting an aniline of formula (II) with an aldehyde of formula (XXV) 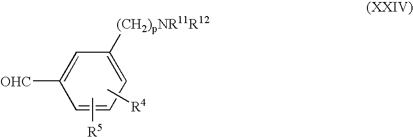
under reducing conditions.

Both reactions may conveniently take place in the presence of a solvent such as an alcohol e.g. methanol or ethanol using for example a hydride reducing agent such as an alkali or alkaline earth metal borohydride (e.g. sodium borohydride or sodium cyanoborohydride). The reactions may be carried out at a temperature in the range from 0° to 60° C., conveniently at room temperature.

Compounds of general formula (I) in which R², R³, R⁴ and R⁵ have a particular meaning may be converted into another compound of the invention by standard methods of interconversion.

For instance, when R² and/or R³ represents a hydroxy or alkoxy group and/or when R⁴ and/or R⁵ represents hydroxy or alkoxy these groups may be interchanged by standard methods of O-alkylation or O-dealkylation. Thus, for example, a compound in which R⁴ represents hydroxy may be prepared by treating a corresponding compound in which R⁴ represents methoxy with a reagent system capable of removing the methyl group e.g. a mercaptide such as sodium ethylmercaptide in a solvent such as dimethylformamide, lithium iodide in collidine, boron tribromide in a halohydrocarbon solvent e.g. methylene chloride or molten pyridine hydrochloride.

When R² represents a hydroxymethyl group this may be converted by oxidation into a corresponding compound of general formula (I) in which R² represents a group COR⁶ (where R⁶ is a hydrogen atom) or CO₂H. Thus, for example, oxidation may be effected using a suitable oxidising agent such as a manganese oxidising agent (e.g. manganese dioxide) in a solvent such as an ether (e.g. 1,4-dioxan) at a suitable temperature up to reflux, a chromium oxidising agent (e.g. Jones reagent) or pyridinium dichromate in a suitable solvent such as a halohydrocarbon (e.g. methylene chloride).

When R² represents an aldehyde group this may be converted by oxidation into a corresponding compound of general formula (I) in which R² represents a group CO₂H. Thus, for example, oxidation may be effected using a suitable oxidising agent such as a source of silver (I) (e.g. silver nitrate) in aqueous alkali optionally in the presence of a cosolvent such as an alcohol (e.g. methanol).

Intermediates of formula (II) may be prepared by reduction of a compound of formula (XIII) 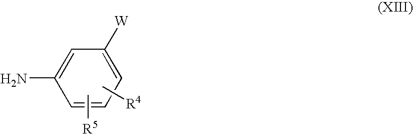
(where W is as defined in formula (XII)) under the reducing conditions described for process (4).

Compounds of formula (XIII) may be prepared by reduction of the corresponding nitro compounds of formula (XIV)

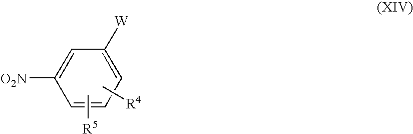

Suitable reducing conditions include, for example, catalytic hydrogenation using a metal catalyst such as palladium oxide on a support such as charcoal, optionally in a solvent such as an alcohol (e.g. ethanol) or an ether (e.g. tetrahydrofuran). Under such conditions, the group W may also be reduced and hence the intermediates of formula (II) may be prepared directly from the compounds of formula (XIV) without prior isolation of the compounds of formula (XIII).

The nitro compounds of formula (XIV) may be prepared from the corresponding halo compounds of formula (XV) 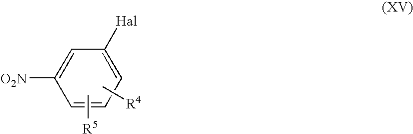
(where Hal is bromine or iodine) using standard methodology.

Intermediates of formula (IV) may be prepared by the following reaction sequence: 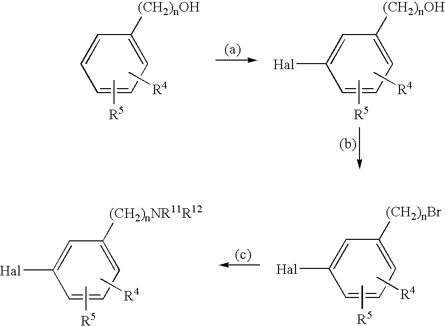
Step (a) is carried out using suitable halogenating conditions, for example, when Hal represents iodine the iodine atom may be introduced using iodine monochloride in a solvent such as methylene chloride;
step (b) is carried out under standard brominating conditions such as using phosphorous tribromide in a halohydrocarbon solvent or using carbon tetrabromide in the presence of triphenylphosphine; and
step (c) is carried out using an amine R¹¹R¹²NH in a suitable solvent such as ethanol, preferably in the presence of a base;
   with the proviso that either R⁴ or R⁵ is a directing group (i.e. fluorine, chlorine, hydroxy, C₁₋₆alkoxy or C₁₋₆alkyl) in a position either ortho or para to the group —(CH₂)ₙOH.

Intermediates of formula (V) may be prepared by reaction of a compound of formula (XIa) or (MIb) with a compound of formula (XVIa) or (XVIb), respectively, 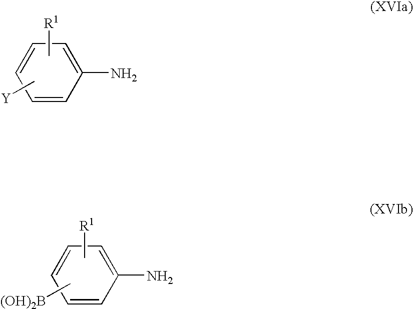
according to the method of general process (3).

Intermediates of formula (II) or (V) may also be prepared from the corresponding carboxylic acid of formula (IX) or (VIII), respectively, using conventional procedures (e.g. by Curtius rearrangement).

Intermediates of formula (Xa) and (Xb), in which X is —CONH—, may be prepared by reaction of a compound of formula (II) with a compound of formula (XVIIa) or (XVIIb), respectively, 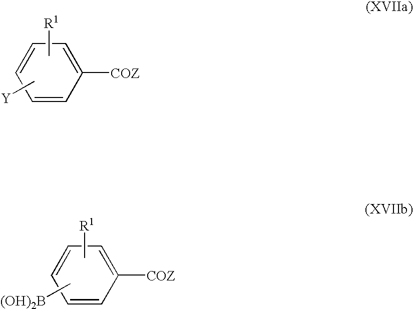
according to the method of general process (2).

Intermediates of formulae (Xa) and (Xb), in which X is —NHCO—, may be prepared by reaction of a compound of formula (IV) with a compound of formula (XVIa) or (XVIb), respectively, according to the method of general process (1).

Intermediates of formula (XII), in which X is —CONH—, may be prepared by reaction of a compound of formula (XIII) with a compound of either formula (III) or (VI) according to the method of general process (1) or (2), respectively.

Alternatively, intermediates of formula (XII), in which W is —CH═CHCHO, —CH═CHCN, —CH═CHCH₂NR¹¹R¹² or —C≡CCH₂NR¹¹R¹², may be prepared from a compound of formula (XVIII) 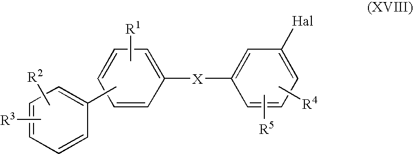
(wherein Hal is the only bromine or iodine atom in the molecule) by reaction with an alkene: H₂C═CHCHO, H₂C═CHCH₂NR¹¹R¹² or H₂C═CHCN; or an alkyne: HC≡CCH₂NR¹¹R¹².

The reaction may be effected in the presence of a palladium reagent and preferably in the presence of a base. The palladium reagent may be, for example, a palladium salt derived from an organic acid (e.g. an acetate) or derived from an inorganic acid (e.g. a chloride or bromide), a palladium complex such as a triarylphosphine palladium complex (e.g. triphenylphosphine or tri(2-methylphenyl)phosphine palladium complex), or a finely divided palladium metal such as palladium on charcoal. The triarylphosphine palladium complex may be generated in situ by reacting a palladium salt (e.g. palladium acetate) with the appropriate triarylphosphine.

Suitable bases include tertiary amines (e.g. triethylamine or tri-n-butylamine) or alkali metal (e.g. sodium or potassium) carbonates, bicarbonates and acetates.

The reaction may be effected in the presence or absence of a solvent. Suitable solvents include nitriles (e.g. acetonitrile), amides (e.g. dimethylformamide, N-methylpyrrolidinone) and water. The reaction may conveniently be carried out at a temperature between room temperature and 200° C., preferably between 50° C. and 160° C.

Compounds of formula (XVIII) may be prepared by the reaction of a compound of formula (V) or (VI) with a compound of formula (XIX) or (XX), respectively, 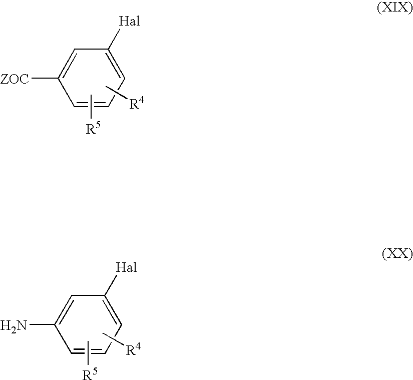
according to the method of general process (2).

Alternatively, intermediates of formula (XII) in which W is —(CH₂)ₚ₋₁CN or —(CH₂)ₚ₋₁CHO may be prepared by the reaction of a compound of formula (III) or (V) with a compound of formula (XXI) or (XXII), respectively, 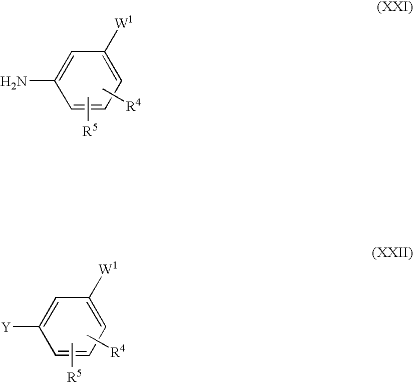
(wherein W¹ represents —(CH₂)ₚ₋₁CN or —(CH₂)ₚ₋₁CHO), according to the method of general process (1).

Intermediates of formulae (XXI) and (XXII) in which W¹ contains a nitrile group may be prepared from the corresponding halo (e.g. bromo) compound using standard methodology.

It will be appreciated that, where necessary, a halogen substituent may be converted into a carboxyl group using standard methodology thus, for example, compounds of formula (VIII) or (IX) may be prepared from an intermediate of formula (III) or (IV), respectively, by lithiation using, for example, n-butyl lithium followed by quenching with carbon dioxide.

The boronic acid intermediates of formulae (Xb), (XIa), (XVIb) and (XVIIb) or their esters, anhydrides or salts may be used in situ under the conditions described above for general process (3).

The aldehydes of formula (XXIII) or (XXIV) may be prepared from an intermediate of formula (IV) or (III), respectively, by lithiation using, for example, n-butyl lithium followed by formylation using, for example, dimethylformamide.

Intermediates of formulae (III), (XIa), (XIb), (XV), (XVIa), (XVIb), (XVIIa), (XVIIb), (XIX) and (XX) are either known compounds or may be prepared by standard methodology or methods analogous to those described herein.

Physiologically acceptable acid addition salts of the compounds of general formula (I) may be prepared by treating the corresponding free base with a suitable acid using conventional methods. Thus, for example, a generally convenient method of forming the acid addition salts is to mix appropriate quantities of the free base and the acid in an appropriate solvent e.g. an alcohol such as ethanol or an ester such as ethyl acetate.

Inorganic basic salts of compounds of general formula (I) may be prepared by treating the corresponding acid of general formula (I) (i.e. a compound of general formula (I) in which R² and/or R³ represents the group CO₂H) with a suitable base using conventional methods.

The invention is illustrated but not limited by the following examples in which temperatures are in ° C. Thin layer chromatography (T.l.c.) was carried out on silica plates. ‘Dried’ refers to drying using sodium sulphate or magnesium sulphate unless otherwise stated. Flash column chromatography (FCC) was carried out on silica gel (Merck 9385) unless otherwise stated, Short path column chromatography (SPC) was carried out on silica gel (Merck 7747) unless otherwise stated.

The following solvent systems were used: System A—dichloromethane:ethanol:0.88 ammonia; System B—dichloromethane:ethanol; System C—hexane:diethyl ether; System D—dichloromethane:hexane; System E—ethyl acetate:ethanol:triethylamine; System F—dichloromethane:methanol:0.88 ammonia; System G—chloroform:methanol:0.88 ammonia.

The following abbreviations are used: ether—diethyl ether; DMF—dimethylformamide; ThF—tetrahydrofuran; AIBN—azobisisobutyronitrile; DME—1,2-dimethoxyethane.

### Intermediate 1

### (a) 3-[3-(Dimethylamino)-1-propynyl]-4-methoxybenzoic acid

A mixture of 3-iodomethoxybenzoic acid (400 mg), N,N-dimethyl-2-propynamine (0.22 ml), copper (I) iodide (14 mg), dichlorobis(triphenylphosphine)palladium (II) (31 mg), triethylamine (3 ml) and DMF (2 ml) was stirred at reflux under nitrogen for 5 h. When cool, the mixture was evaporated and the residue purified by FCC using gradient elution with System A (134:60:6 to 50:45:5) to afford a solid which crystallised from ethanol to give the title compound (85 mg) as fine cream crystals, m.p. 167-169.5°.

Similarly prepared was:

### (b) 4-Methoxy-3-[3-(methylamino)-1-propynyl]benzoic acid

(1.97g), T.l.c. (System A 10:8:1) Rf 0.25

From 3-iodo-4-methoxybenzoic acid (3.0 g) and N-methyl-2-propynamine (2.24 g) using acetonitrile as reaction solvent. The title compound was obtained without crystallisation.

### Intermediate 2

### 3-[3-(Dimethylamino)propyl]-4-methoxybenzoic acid

A solution of Intermediate 1(a) (2.81 g) in DMF (40 ml) and ethanol (20 ml) was added to a pre-reduced suspension of 10% palladium oxide-on-carbon (1.5 g) in ethanol (20 ml) and hydrogenated at room temperature and pressure until uptake ceased. The catalyst was filtered off, the filtrate evaporated, and the residue crystallised from acetonitrile (45 ml) to give the title compound (1.90 g) as fine white crystals, m.p. 135-137.5°.

### Intermediate 3

### 5-Iodo-2-methoxybenzenepropanol

A solution of iodine monochloride (17.0 g) in methylene chloride (55 ml) was added dropwise, under nitrogen, over a period of 0.5 h to a stirred, ice-cooled solution of 2-methoxybenzenepropanol (10.0 g) in methylene chloride (50 ml). The dark solution was stirred, with ice cooling for 1.25 h and then washed with 20% aqueous sodium thiosulphate solution. The pale yellow organic solution was then dried and concentrated in vacuo to give the title compound (16.38 g) as a pale pink solid, m.p. 68-70°.

### Intermediate 4

### 2-(3-Bromopropyl)-4-iodo-1-methoxybenzene

A solution of triphenylphosphine (18.5 g) in methylene chloride (120 ml) was added dropwise under nitrogen to a stirred, cooled solution of Intermediate 3 (13.3 g) and carbon tetrabromide (19.2 g) in methylene chloride (200 ml). The orange solution was stirred, with external cooling, for 2 h and then concentrated in vacuo. Purification by FCC of the residual solid (Merck 7734) eluting with hexane afforded the title compound (15.35 g) as a colourless oil.

T.l.c. (hexane) Rf 0.39.

### Intermediate 5

### 5-Iodo-2-methoxy-N,N-dimethylbenzenepropanamine hydrochloride

A solution of Intermediate 4 (500 mg) in a mixture of 33% ethanolic dimethylamine (10 ml) and absolute ethanol (2 ml) was stirred at reflux under nitrogen for 2 h, allowed to cool, and then concentrated in vacuo. Purification by FCC of the residual white solid eluting with System A (200:10:1) afforded a colourless oil which contained the free base of the title compound (272 mg) as a white solid. This material was dissolved in refluxing absolute ethanol (1 ml) and ethereal hydrogen chloride (0.5 ml) was added. The stirred solution was diluted with dry ether (10 ml) and the resultant solid filtered off, washed with dry ether and dried in vacuo at 60° for 20 h to give the title compound (195 mg) as a white solid, m.p. 143-144°.

### Intermediate 6

### [4-[Bis(phenylmethyl)amino]phenyl]boronic acid

n-Butyllithium (1.60M in hexane, 98 ml) was added dropwise under nitrogen to a stirred solution of N-(4bromophenyl)-N-(phenylmethyl)benzenemethanamine (50.0 g) in dry ThF (500 ml) at −67 to −65° over 30 min. After 1 h, triisopropylborate (57 ml) was added dropwise over 20 min and the mixture stirred at 23° for 16 h. Water (80 ml) was added, the mixture was evaporated, and then co-evaporated with ethanol (2×100 ml). The residue was treated with dichloromethane (200 ml) and the slurry purified by FCC using gradient elution with a gradient of System B (1:0) to (97:3) to afford the title compound (5.6 g) as fine cream crystals.

T.l.c. System B (50:1) Rf 0.16.

### Intermediate 7

### 3′-Amino[1,1′-biphenyl]-4-carboxamide

A solution of 4-bromobenzamide (2.15 g) and 3-aminophenylboronic acid hemisulphate (2.00 g) in DME (100 ml) was treated with a solution of sodium carbonate (1.71 g) in water (50 ml), followed by tetrakis(triphenylphosphine)palladium (0) (496 mg) and then stirred at reflux under nitrogen for 3 h. When cool, the mixture was evaporated, treated with aqueous 2M sodium carbonate (200 ml) and extracted with ethyl acetate (4×120 ml). The combined, dried organic extracts were evaporated and the residue crystallised from ethanol to give the title compound (1.03 g) as fine cream crystals, m.p. 206-208°.

Similarly prepared was:

### Intermediate 8

### (a) 4′-[Bis(phenylmethyl)amino][1,1′-biphenyl-3-carboxamide

(4.00 g), as fine white needles, T.l.c. (ether) Rf 0.11.

From 3-bromobenzamide (4.63 g) and Intermediate 6 (7.34 g) with recrystallisation from ethyl acetate.

(b) 4′-[Bis(phenylmethyl)amino]-N,N-dimethyl[1,1′-biphenyl]-4-sulphonamide

(0.74 g) as fine white needles, m.p. 181.5-185.5°.

From Intermediate 6 (5.00 g) and 4-bromo-N,N-dimethyl-benzenesulphonamide (4.16 g) except that 1M sodium carbonate (200 ml) was used in the work-up procedure.

### (c) 4′-[Bis(phenylmethyl)amino][1,1′-biphenyl-4-carboxamide

(6.44 g) as fine white needles, m.p. 199-200.5°.

From Intermediate 1 (8.00 g) and 4-bromobenzamide (5.04 g) except that the residue was suspended in 1M sodium carbonate (150 ml), the solid filtered off, dried and treated with refluxing acetonitrile (600 ml). The mixture was then filtered, the filtrate heated to reflux and the precipitate, which resulted upon cooling, filtered off. The mother liquors were evaporated and the residue crystallised from acetonitrile to give a further crop of the title compound (1.78 g).

### (d) 4′-[Bis(phenylmethyl)amino-N,N-dimethyl[1,1′-biphenyl]-4-carboxamide

(3.48 g) as white crystals, m.p. 133-135° (softens 86°).

From Intermediate 6 (5.00 g) and 4-bromo-N,N-dimethylbenzamide (3.72 g) except that the product was crystallised from ethanol:ethyl acetate (1:2). Further recrystallisation of the mother liquors gave the title compound (1.82 g) as white crystals.

### (e) 4′-(Bis(phenylmethyl)amino]-3-hydroxy[1,1′-biphenyl]-4-carboxylic acid

(2.23 g) as a crystalline solid, m.p. 224-2280 (dec).

From 2-hydroxy-4-iodobenzoic acid (2.11 g) and Intermediate 6 (2.54 g) with a reaction time of 30 h after which the mixture was cooled and filtered, washing well with water. The filtrate was acidified to pH6 with 2N sulphuric acid and extracted with ethyl acetate (3×100 ml). The combined extracts were dried and evaporated to give a yellow solid (3.58 g). FCC using dichloromethane:acetic acid (100:1) as eluent, gave the title compound.

### (f) 1-[4′-[bis(phenylmethyl)amino][1,1′-biphenyl]-4-yl]ethanone

(2.01 g) as fine cream-coloured crystals, m.p. 140-143°.

From Intermediate 6 (4.00 g) and 1-(4bromophenyl)ethanone (2.51 g). The residue was crystallised from ethyl acetate to give a crop of impure product (3.76 g) and the mother liquors were concentrated to give a second crop (0.66 g). The first and second crops were combined and purified by FCC eluting with System C (9:17:3) to afford the title compound.

### (g) 1-[4′-Bis(phenylmethyl)amino][1,1′-biphenyl]-2-yl]ethanone

(1.07 g) as a yellow gum. T.l.c. (dichloromethane) Rf 0.51.

From Intermediate 6 (0.96 g) and 2-bromoacetophenone (0.72 g) with purification by FCC eluting with System D (3:7) followed by (1:1).

### (h) N-(4′-Methoxy[1,1′-biphenyl]-4-yl)-N-phenylmethyl-benzenemethanamine

(1.58 g) as a colourless crystalline solid, m.p. 100-102°.

From Intermediate 6 (1.903 g) and 4-iodo-1-methoxybenzene (1.404 g). Except that after 4.5 h the reaction mixture was cooled, poured into water (200 ml), and extracted with ether (500 ml and 100 nm). The combined extracts were dried and evaporated to give a brown oil, which was purified by FCC, using ethyl acetate:hexane (1:50 followed by 1:20) as eluents to give the title compound.

### (i) 4′-(Aminocarbonyl)[1,1′-biphenyl]-4-carboxylic acid

(441 mg), T.l.c. (ethyl acetate) Rf 0.27.

From 4-bromobenzamide (777 mg) and 4-boronobenzoic acid (677 mg). When cool, the reaction mixture was evaporated, and treated with aqueous 2M-hydrochloric acid (60 ml). The solid was filtered off, dried in vacuo over phosphorus pentoxide, and then crystallised from acetic acid to give the title compound.

### Intermediate 9

### 4′-Amino-methyl[1,1-biphenyl]-2-methanol

A solution of Intermediate 8(g) (5.60 g) in 2-methoxyethanol (200 ml) was added to a suspension of wet 10% palladium on carbon (2.5 g) in 2-methoxyethanol (50 ml) and the reaction mixture was hydrogenated at room temperature and pressure for 16 h. The catalyst was filtered off and the filtrate was adsorbed onto silica gel. The residue was purified by FCC eluting with System C (3:1) followed by (1:3) to give the title compound (2.55 g) as an off-white solid, m.p. 108-112°.

Similarly prepared was:

### Intermediate 10

### 4′-Amino-3-hydroxy[1,1′-biphenyl]-4-carboxylic acid

(981 mg), m.p. 212-214° dec.

From Intermediate 8(e) (2.16 g) using 2-methoxyethanol as solvent. The crude product (1.23 g) was crystallised from aqueous methanol to give the title compound. Concentration of the mother liquors gave a second crop (133 mg).

### Intermediate 11

### 4′-Amino-N,N-dimethyl[1,1′-biphenyl]-4-carboxamide

A solution of Intermediate 8(d) (5.21 g) in ThF (100 ml) was added to a suspension of 10% palladium on carbon (2.40 g) in ethanol (30 ml) and the mixture hydrogenated at room temperature and pressure for 20 h. The catalyst was filtered off and the solution evaporated to give cream crystals. Recrystallisation of the crystals from ethyl acetate-tetrahydrofuran (1:1) gave the title compound (1.58 g) as white crystals, m.p. 184.0-186.5°. Recrystallisation of the mother liquors gave a further crop of the title compound (274 mg) as beige crystals.

### Intermediate 12

### 4′-Amino[1,1′-biphenyl]-4-carboxamide

A solution of Intermediate 3 (7.75 g) in dry THF (200 ml) was added to a mixture of pre-reduced 10% palladium oxide-on-carbon (3.00 g) and the stirred suspension hydrogenated at room temperature and pressure until uptake ceased. The catalyst was filtered off, the filtrate evaporated, and a solution of the residue in DMF (150ml) was added to a mixture of pre-reduced 10% palladium oxide-on-carbon (2.00 g) in ethanol (25 ml). The stirred suspension was hydrogenated at room temperature and pressure until uptake ceased. The catalyst was filtered off and the filtered pad washed with hot ethanol. The filtrate and washings were evaporated to give a solid (4.00 g). A portion of the solid (500 mg) was recrystallised from acetonitrile to give the pure title compound (56 mg) as fine light grey crystals, m.p. 281-284°.

### Intermediate 13

### 4′-Amino-α-methyl[1,1′-biphenyl]-4-methanol

A solution of Intermediate 8(f) (1.80 g) in dry THF (22 ml) was added to a suspension of 10% palladium oxide-on-carbon (0.9 g) in dry THF (15 ml) and the stirred mixture hydrogenated at room temperature and pressure. The catalyst was filtered off and the filtrate evaporated and purified by FCC. Elution with System C (3:10:1) afforded the title compound (645 mg) as fine white crystals, m.p. 111-115°.

Similarly prepared were:

### Intermediate 14

### (a) 4′-Ethyl[1,1′-biphenyl]-4-amine

(84 mg), m.p. 67-77°.

From a more extensive hydrogenation with 10% palladium oxide on carbon of Intermediate 8(f) (1.80 g).

### (b) 4′-Amino-3-hydroxy[1,1′-biphenyl]-4-carboxylic acid

(981 mg) as light brown needles, m.p. 212-214° dec.

From Intermediate 8(e) (2.16 g) with purification by crystallisation from aqueous methanol. Concentration of the mother liquors gave a second crop (133 mg).

### Intermediate 15

### 4′-Amino-N,N-dimethyl[1,1′-biphenyl]-4-sulphonamide

A solution of Intermediate 8(b) (4.28 g) in DMF (100 ml) was added to pre-reduced 10% palladium oxide-on-carbon (2.00 g) in ethanol (30 ml) and the stirred suspension was hydrogenated at room temperature and pressure until uptake ceased. The catalyst was filtered off, the filtrate evaporated, and the residue purified by SPC. Elution with dichloromethane afforded a solid which crystallised from ethanol to give the title compound (916 mg) as fine white needles, m.p. 178.5-180°. The mother liquors were concentrated to give a further crop of the title compound (176 mg). Further elution of the column gave another crop of the title compound (775 mg).

Similarly prepared were:

### Intermediate 16

### (a) 4′-Amino[1,1′-biphenyl]-3-carboxamide

(1.28 g), m.p. 187.5-190°.

From Intermediate 8(a) (3.99 g), with purification by crystallisation from ethanol. The mother liquors were evaporated to give a second crop of the title compound (551 mg).

T.l.c. (ethyl acetate:hexane, 1:1) Rf 0.04.

### (b) 4-Methoxy-3-[3-(methylamino)propyl]benzoic acid hydrochloride

(2.10 g) m.p. 195-198° C.

From Intermediate 1(b). The catalyst was filtered off and the solvent was then evaporated to give the title compound.

### Intermediate 17

### [4-(Methylsulphinyl)phenyl]boronic acid

A solution of (4-(methylthio)phenyl]boronic acid (1.008 g) in acetonitrile (60 ml) and water (6 ml) was cooled with a dry ice-acetone bath until it just began to freeze. A solution of ceric ammonium nitrate (6.72 g) in water (10 ml) was added with swirling. The resulting solution was left to warm to room temperature. The mixture was then basified to pH5 by addition of 8% aqueous sodium bicarbonate (16 ml) and evaporated to dryness by re-evaporation with absolute ethanol. The dried residue was purified by SPC (Merck 7729) using ethyl acetate:ethanol (9:1 followed by 4:1 and 7:3) as eluent, to give impure product (2.6 g) as a colourless solid. The solid was further purified by FCC eluting with System B (9:1) to give the title compound (1.004 g) as a colourless gum.

T.l.c. System B (9:1) Rf 0.35.

The gum was dissolved in warm water and the solution evaporated to give a colourless crystalline solid (847 mg). Recrystallisation from water (15 ml) gave a further crop of the title compound (697 mg) as almost colourless needles.

Analysis Found: C, 45.6; H, 4.85; S, 17.55. C₇H₉BO₃S requires: C, 45.7; H, 4.95; S, 17.4%.

### Intermediate 18

### 5-Bromo-2-methoxy-N,N-dimethylbenzeneethanamine

Dimethylamine (30% w/v in industrial methylated spirits, 20 ml) was added to a stirred solution of 4-bromo-2-(2-bromoethyl)-1-methoxybenzene (2.95 g) in ethanol (10 ml) and THF (5 ml) and heated at reflux for 1.5 h. The cooled solution was evaporated, treated with aqueous saturated sodium bicarbonate (50 ml), and extracted with ethyl acetate (5×50 ml). The combined, dried organic extracts were evaporated, and the residue purified by FCC using gradient elution with System A (967:30:3 to 945:50:5) to afford the title compound (1.90 g) as a pale yellow oil.

T.l.c. System A (945:50:5) Rf 0.21.

### Intermediate 19

### 3-[2-(Dimethylamino)ethyl]-4-methoxybenzoic acid hydrochloride

Tert-butyllithium (1.8M in pentane, 5.75 ml) was added dropwise under nitrogen at −78° to a stirred solution of Intermediate 18 (1.485 g) in dry TBF (15 ml) and stirring was continued for 15 min at −78°. Carbon dioxide was passed through the solution for 15 min and then the reaction was stirred at 230 for 1 h with carbon dioxide passing through it. The mixture was evaporated and the residue purified by FCC using gradient elution with System A (89:10:1 to 50:45:5) to afford a white solid (1.315 g). A portion of this (220 mg) was recrystallised from ethanol (2.5 ml) to give the title compound (51 mg) as fine white crystals.

T.l.c. System A (50:45:5) Rf 0.1.

### Intermediate 20

### N-(4-Bromophenyl)-3-[3-(dimethylamino)propyl-4-methoxybenzamide

### (i) 3-[3-(Dimethylamino)propyl]-4-methoxybenzoyl chloride, hydrochloride

Thionyl chloride (3.5 ml, 5.7 g) and dry DM (2 drops) was added to Intermediate 2 (1.5 g) and the mixture heated on a steam bath for 5 min. The excess thionyl chloride was evaporated under reduced pressure whilst heating and the solid residue which formed re-evaporated with dry toluene (1 ml×2) then dried in vacuo to give the title compound as a buff powder (1.88 g).

### (ii) N-(4Bromophenyl)-3-[3-(dimethylaminopropyl]-4-methoxybenzamide

The product of step (i) (5.84 g) was treated with 4-bromobenzamine (3.4 g) and dry pyridine (20 ml) and heated at reflux under nitrogen for 1.5 h. When cool, water (20 ml) was added, followed by solid sodium carbonate (1 g) and the solution evaporated in vacuo affording a grey-white residue which was washed with water, filtered, air-dried, and recrystallised from ethyl acetate to give the title compound as a cream solid (3.93 g) m.p. 145.5-146° C.

Evaporation of the filtrate yielded further solid, which was re-crystallised from ethyl acetate to give a second crop of crystals (1.79 g) m.p. 145.5-146° C.

### Intermediate 21

### N-(4-Bromophenyl)-3-[3-(dimethylamino)propyl]-4-hydroxybenzamide

Intermediate 20 (1.32 g) was added to a solution of boron tribromide in dichloromethane (1M; 112.8 ml under nitrogen and the mixture stirred for 5 h before adding methanol (20 ml). The solvent was evaporated in vacuo and the residue purified by FCC eluting with System A (189:10:1) to give the title compound (700 mg) as a white powder.

| | | | |
|---|---|---|---|
| Analysis Found: | C, 57.2; | H, 5.7; | N, 7.2. |
| C₁₈H₂₁BrN₂O₂ requires | C, 57.3; | H, 5.6; | N, 7.4%. |

### Intermediate 22

### 4′-Amino-3-hydroxy[1,1′-biphenyl]-4-methanol

A stirred suspension of lithium aluminium hydride (500 mg) in dry, redistilled ThF (50 ml) under nitrogen was treated with Intermediate 10 (500 mg). The resulting green suspension was stirred at room temperature for 48 h, quenched by careful addition of water, and diluted with saturated aqueous ammonium chloride (300 ml), and extracted with ether (5×300 ml). The combined extracts were dried and evaporated to give a light yellow brown solid (315 mg). crystalisation from ethyl acetate-hexane gave the title compound (214 mg) as light brown needles, T.l.c. ethyl acetate Rf 0.50.

### Intermediate 23

### [4-[[(1,1-Dimethylethyl)dimethylsilyl]oxy]phenyl]boronic acid, bimolecular anhydride

n-Butyl lithium (1.6M; 9.7 ml) was added, over 8 min, under nitrogen to a stirred, cooled (−70°) solution of 1-bromo4-[[(1,1-dimethylethyl)dimethylsilyl]oxy]benzene (4 g) in dry THF (40 ml). After a further 25 min, the resulting solution was added over 10 min to a stirred cooled (−66°) solution of tri-isopropyl borate (10 ml) in TEF (40 ml) and the mixture stirred at room temperature for 2 h. Water (10 ml) and, after a further 5 min, pH 6.5 phosphate buffer (100 ml) and ether (50 ml) were added and the mixture stirred vigorously for 10 min. The aqueous layer was extracted with ether (2×70 ml) and the combined organic solutions dried and evaporated in vacuo to leave a white solid. Crystallisation from ether gave the title compound (2.02 g) as a white solid m.p. 193-7°.

T.l.c. (dichloromethane:methanol, 96:4) Rf 0.56

### Intermediate 24

### N-[(4′-Nitro[1,1′-biphenyl]-4-yl)carbonyl]glycine ethyl ester

To 4′-nitro[1,1-biphenyl]-4-carboxylic acid (3.65 g) was added thionyl chloride (10 ml) and DMF (0.2 ml) and the mixture heated on a steam bath. The excess thionyl chloride was evaporated in vacuo and the residue re-evaporated with toluene (20 ml) then ether (20 ml×2). The solid residue was suspended in pyridine (20 ml) and glycine ethyl ester hydrochloride (2.30 g) added and the mixture heated on a steam bath until complete solution had occurred. The solution was stood at room temperature for 45 min then poured into water (300 ml) and then acidified with 5N HCl. The mixture was extracted with ethyl acetate (150 ml×4) and the combined extracts dried and evaporated in vacuo to give a red residue which was suspended in cold ether and filtered to give a solid which was washed with ether and dried to give the title compound (4.606 g) m.p. 145-148°.

### Intermediate 25

### N-[(4′-Amino[1,1′-biphenyl]-4-yl)carbonyl]glycine ethyl ester maleate (1:1)

To a hot suspension of Intermediate 24 (1.49 g) in ethanol (70 ml) was added Raney Nickel (1 g) and hydrazine hydrate (1.2 ml) in 0.2 ml portions dropwise during 20 min with stirring. The mixture was heated on a steam bath and after the effervescence subsided, was heated for a further 20 min. The suspension was filtered in a stream of nitrogen and the resulting suspension was evaporated to dryness and the residue extracted with hot ethyl acetate (100 ml) and filtered, and the filtrate evaporated to dryness. The residue was suspended in cold ether, filtered, and the residue dried to give the free base of the title compound (0.963 g) m.p. 174-179°. To a solution of the free base (0.1 g) in ethyl acetate was added a solution of maleic acid (0.04 g) in ethyl acetate. The suspension was filtered, the residue washed with ethyl acetate and dried to give the title compound (0.108 g) m.p. 149-151°.

### Intermediate 26

### (g)-3-(2-Methoxy-5-nitrophenyl-2-propenenitrile mixture with (Z) (1:1)

A solution of 2-iodo-1-methoxy-4-nitrobenzene (500 g), acrylonitrile (0.14 ml), triethylamine (0.62 ml) and acetonitrile (1.2 ml), was treated with palladium acetate (28 mg) and heated at 1100 for 16 h. When cool the mixture was poured into aqueous saturated sodium bicarbonate (30 ml), extracted into ethyl acetate (3×30 ml), and the combined, dried organic extracts were evaporated and purified by SPC and elution with System C (4:1 to 7:3) afforded the title compound (128 mg) as white crystals.

T.l.c. System C (2:1) Rf 0.11

### Intermediate 27

### 5-Amino-2-methoxy-N,N-dimethylbenzenepropanamine

A solution of Intermediate 26 (4.33 g) in dry THF (80 ml) and ethanolic dimethylamine (33% w/v, 80 ml) was added to a suspension of pre-reduced 10% palladium oxide on carbon (2.00 g) in ethanol (30 ml) and the stirred mixture hyrogenated at room temperature and pressure for 5 h. The catalyst was filtered off and the filtrate evaporated. A solution of the filtrate in ethanolic dimethylamine (100 ml) was added to a suspension of pre-reduced 10% palladium oxide on carbon (2.00 g) in ethanol (30 ml) and the stirred mixture hydrogenated at room temperature and pressure for 16 h. The catalyst was once again replenished as above and hydrogenation continued for 70 h. The catalyst was filtered off and the filtrate was purified by SPC with gradient elution using System A (945:50:5 to 912:80:8) to afford the title compound (3.20 g) as a light brown oil.

T.l.c. System A (89:10:1) Rf 0.12.

### Intermediate 28

### (4-Formyl-2-methylphenyl)boronic acid

A stirred solution of 2,5-dibromotoluene (5.00 g) in dry TBF (200 ml) under nitrogen at −78° was treated dropwise with n-butyllithium (11.8 ml, 1.69M in hexane). After 1 h, DMF (1.6 ml) was added and the mixture was allowed to warm to 0° and then recooled to −78° and treated with more n-butyllithium (11.8 ml, 1.69M in hexane). After 1 h, triisopropylborate (7.0 ml) was added and the cooling bath removed. After 1 h, 2N hydrochloric acid (80 ml) was added and the THF removed under vacuum. The residue was extracted with ether (3×100 ml) and the combined extracts were dried and evaporated to give an oil. SPC (Merck 7729) using chloroform:ethanol (50:1) as eluent gave the product (2.21 g) as an almost colourless oil. Addition of acetone (50 ml) and water (50 ml) and evaporation to dryness gave the title compound (1.31 g) as a colourless solid.

T.l.c. (chloroform:ethanol 50: 1) Rf 0.25.

### Intermediate 29

### 4′-Methoxy[1,1′-biphenyl]-4-amine, hydrochloride

A stirred mixture of Intermediate 8(h) (1.50 g), 10% palladium oxide on carbon (400 mg of 50% w/w wet material), and 2N hydrochloric acid (3 ml) in ethanol (40 ml) was hydrogenated at room temperature and pressure. After 3 h the reaction mixture was filtered and the filtrate was evaporated to dryness to give an almost colourless solid. Crystallisation from absolute ethanol gave the title compound (543 mg) as almost colourless plates, m.p. 232-234° dec.

### Intermediate 30

### [4-[[3-[3-(Dimethylamino)propyl]-4-methoxybenzoyl]amino]phenyl boronic acid

A stirred solution of Intermediate 20 (3 g) in dry THF (125 ml), under nitrogen at −78° C. was treated dropwise with n-butyllithium (10.1 ml of a 1.52M solution in hexane). After 1 h, triisopropylborate (4.4 ml) was added dropwise at −78° C., and the reaction was allowed to stir for 30 min. The cooling bath was then removed, and the reaction was stirred under nitrogen at 28° C. for 17 h. Hydrochloric acid (2N; 4 ml) and water (50 ml) were added, the reaction mixture evaporated to dryness, and purified by FCC eluting with System A (75:23:2) to give the title compound as a cream foam (1.14 g).

T.l.c. System A (60:40:4) Rf 0.26

### Intermediate 31

### 4-(Bromomethyl)-2-chloro-1-iodobenzene

A mixture of 2-chloro-1-iodo-4-methylbenzene (1 g), N-bromosuccinimide (0.78 g), AIBN (25 mg), and carbon tetrachloride (15 ml), was heated at reflux using a 150 W light bulb for 4 h. When cool, the reaction mixture was evaporated in vacuo and the residue was purified by FCC eluting with hexane to give the title compound as a colourless oil (0.833 g).

T.l.c. hexane:ethyl acetate (20:1) Rf 0.65.

### Intermediate 32

### 3-chloro-4-iodobenzene methanol

A mixture of Intermediate 31 (2 g), potassium acetate (0.93 g) and ethanol (95%, 10 ml) was heated at reflux for 12 h. When cool, the reaction mixture was filtered and the filtrate was treated with potassium hydroxide (0.41 g), and allowed to reflux under nitrogen for 16 h. When cool, the reaction mixture was evaporated to dryness, and the resultant yellow solid was purified by FCC, eluting with hexane:ethyl acetate (8:1), to give the title compound as a colourless solid (0.97 g) m.p. 80-82° C.

### Intermediate 33

### 4-Bromo-3-methylbenzenemethanol

Diborane in TBF (1M; 220 ml) was added dropwise to a solution of the 4-bromo-3-methylbenzoic acid (20.0 g) in THF (100 ml) at room temperature under nitrogen. The solution was stirred for 5 h, treated cautiously with water (20 ml) and aqueous sodium hydroxide (2N; 200 ml) and extracted with ether (3×100 ml). The dried extract was evaporated to give the title compound as a colourless oil (11.8 g).

T.l.c. System C (1:1) Rf 0.35.

### Intermediate 34

### 1-Bromo-4-(methoxymethyl)-2-methylbenzene

Sodium hydride (80% disp; 60 mg) was added in one portion to a solution of Intermediate 33 (300 mg) and iodomethane (425 mg) in DMF (2 ml) at room temperature under nitrogen. The resulting mixture was stirred for 16 h, treated with water (20 ml) and extracted with ether (2×30 ml). The ether extract was washed with water (25 ml), dried and evaporated to give the title compound as a yellow oil (340 mg).

T.l.c System C (9:1), Rf 0.6.

### Intermediate 35

### 4′-[Bis(phenylmethyl)amino]-2-methyl[1,1′-biphenyl]4-ol hydrochloride

From a hot mixture of 4-bromo-3-methylphenol (2.0 g) and Intermediate 6 (3.4 g) according to the method of Intermediate 7. Purification of a portion of the product (100 mg) was effected by dissolving the product in the minimum quantity of refluxing ethyl acetate and then treating it with ethereal hydrogen chloride to yield the title compound as a white solid (72 mg) m.p 158-162° C. (140° C.-reddens).

Similarly prepared was:

### Intermediate 36

### 4′-[Bis(phenylmethyl)amino][1,1′-biphenyl]-4-carboxamide as fine white needles (6.44 g) m.p. 199-200° C.

From 4-bromobenzamide (5.04 g) and Intermediate 6 (8.00 g) using refluxing acetonitrile instead of ethyl acetate in the purification step.

### Intermediate 37

### 4′-Amino-2-methyl[1,1′-biphenyl]-4-ol

A solution of the free base of Intermediate 35 (2.75 g) in ethanol (65 ml) was added to a pre-hydrogenated mixture of 10% palladium on carbon (2.75 g) in ethanol (10 ml) and the stirred mixture hydrogenated at room temperature for 3 h. The mixture was filtered, washing the filter pad with ethanol (1.25 litres) and the filtrate adsorbed onto silica gel (Merck 7734, 30 ml). Purification by FCC eluting with hexane-ethyl acetate (2:1) gave the title compound as a cream-yellow solid (0.598 g) which was crystallized from refluxing toluene (5 ml) to give the pure title compound as an off-white solid (0.369 g) m.p. 156-157° C.

### Intermediate 38

### 4′-[Bis(phenylmethyl)amino][1,1′-biphenyl]-4-methanamine

A cooled (ice-bath) suspension of lithium aluminium hydride (10 g) in dry THF (125 ml), was treated dropwise with a solution of Intermediate 36 (5.0 g) in dry THF (250 ml) and heated under nitrogen at reflux for 17 h. The mixture was cooled (ice-bath) and treated dropwise with water (1.0 ml) followed by 15% sodium hydroxide solution (1.0 ml) then water (3.0 ml). The resultant solution was evaporated to dryness and purified by SPC, eluting with System A (97:3:0.3). Increasing the polarity to 95:5:0.5 gave the title compound as a cream solid (5.76 g) m.p. 122-125° C.

### Intermediate 39

### N-[4′-[Bis(phenylmethyl)amino][1,1′-biphenyl]methyl]acetamide

Intermediate 38 (4.0 g) in dry pyridine (60 ml), was treated with acetyl chloride (1.14 ml) at 0° C. and stirred for 24 h at room temperature under nitrogen. 8% sodium bicarbonate (120 nm) was added and the mixture evaporated to dryness and the residue purified by FCC eluting with System A (96.5:3.5:0.35) to give the title compound as a yellow solid (4.29 g) m.p. 133-136° C.

### Intermediate 40

### N-[4′-Amino[1,1′-biphenyl]methyl]acetamide

A solution of Intermediate 39 (4.0 g) and 2N HCl (1.9 ml) in DMF (250 ml), was added to a pre-hydrogenated mixture of 10% palladium on carbon (1.7 g, dry catalyst) in DMF (300 ml), and hydrogenated at room temperature and pressure for 5 h. The catalyst was filtered off and washed well with DMF (3 litres) and hot ethanol (2 litres). 8% sodium bicarbonate solution was added (200 ml) and the mixture was evaporated to dryness and purified by FCC eluting with System A (97:3:0.3) to give the title compound as a cream solid (1.29 g) m.p.174-175° C.

### Intermediate 41

### (2-chloro4-methylphenyl boronic acid

A stirred solution of 2-chloro-1-iodo-4-methylbenzene (760 mg) in dry THF (30 ml) under nitrogen at −78° C., was treated dropwise with n-butyl lithium, (2.04 ml of a 1.62M solution in hexane). After 30 min, trisopropylborate (0.84 ml) was added dropwise at −78° C., and the reaction mixture was stirred for 1h. The cooling bath was then removed and the reaction mixture was stirred at 23° C. under nitrogen for 19 h. Aqueous hydrochloric acid (2M, 2.4 ml) was added and after stirring for 30 min, the mixture was extracted with ether (3×20 ml), discarding the aqueous phase. The combined organic extracts were washed with aqueous 2M sodium hydroxide (3×100 ml), and the organic layer was discarded. The aqueous extracts were combined, acidified to pH1 with conc. hydrochloric acid and extracted with ether (3×200 ml). The ethereal fractions were combined, dried and evaporated in vacuo to give the title compound as a grey solid (367 mg) m.p. 187-188° C.

### Intermediate 45

### N-(4-Bromo-3-methylphenyl)-3-[3-(dimethylamino)propyl]-methoxybenzamide

A mixture of the hydrochloride salt of Intermediate 2 (2.00 g) and thionyl chloride (20 ml) was heated at reflux for 1 h. The excess thionyl chloride was evaporated under reduced pressure. A mixture of the resulting solid and 4-bromo-3-methylaniline (1.50 g) in dry pyridin (20 ml) was heated at reflux for 30 min. The solvent was evaporated and the residue adsorbed onto silica gel. The silica gel residue was purified by FCC eluting with System A (150:8:1) to give the title compound as a brown gum (2.82 g).

T.l.c. System A (100:8:1) Rf 0.30

### Intermediate 46

### (4-Acetylphenyl)boronic acid

n-Butyllithium (81.6 ml) was added dropwise to a stirred solution of 2-(4-bromophenyl)-2-methyl-1,3-dioxolane (30.0 g) in dry THF (400 ml), under nitrogen, at −75° C. After a period of 1.5 h, triisopropylborate (31.2 ml) was added dropwise at −75° C. The reaction was allowed to warm to room temperature over a 3 h period. Hydrochloric acid (2N; 200 ml) was added and the reaction was allowed to stand at room temperature overnight. The solvent was evaporated and the residue was purified by FCC eluting with ether to give a white solid which was purified by crystallisation from water (200 ml) to give the title compound (14.52 g) as a white crystalline solid, m.p. 268-270° C.

### Intermediate 47

### 4-(Hydroxymethyl)-2-methylbenzeneboronic acid

n-Butyl lithium (1.67M in hexane, 133 ml) was added dropwise under nitrogen at −65° to −60° to a stirred solution of Intermediate 33 (18.94 g) in dry THF (500 ml) over 30 min. After 1 h at −65°, triisopropylborate (65 ml) was added at −60 to −65° over 30 min. The reaction was stirred at 230 for 1 h and then stood at 23° for 7 days. The mixture was evaporated, treated with aqueous 2N sodium hydroxide (200 ml) and extracted with ethyl acetate (2×200 ml). The organic extracts were discarded and the aqueous phase acidified to pH1 by the addition of conc. hydrochloric acid. The solution was extracted with ethyl acetate (4×200 ml) and the combined, dried organic extracts were evaporated to give the crude title compound as a cream foam (11.02 g).

A portion of this (2.00 g) was heated to reflux in water (8 ml) and decanted from the heavy yellow oil. The liquid was re-heated and decanted from the oil, and this process repeated twice more. On cooling the title compound crystallised as an off-white tacky solid (221 mg).

T.l.c. ether:acetic acid (99:1) major product Rf 0.61.

### Intermediate 48

### 1-[4′-[Bis(phenylmethyl)amino]-2-methyl[1,1′-biphenyl]-4-yl]ethanone

4-Bromo-3-methylacetophenone (3.165 g) and Intermediate 6 (5.189 g), were treated according to the method of Intermediate 35. Purification by by FCC eluting with hexane then hexane:dichloromethane (1:1) gave the title compound as a pale yellow solid (5.3 g), m.p. 145-146° C.

### Intermediate 49

### 1-(4′-Amino-2-methyl[1,1′-biphenyl-4-yl)ethanone

Intermediate 48 (4.5 g) was dissolved in 2-methoxyethanol (150 ml) and added to a pre-reduced suspension of palladium oxide on carbon (10%, 2.25 g) in 2-methoxyethanol (150 ml) and hydrochloric acid (5N; 2 ml), and stirred under 1 atmosphere of hydrogen for 2 h. The catalyst was filtered through celite, and the solvent evaporated in vacuo to give a light brown oil. This oil was preadsorbed onto silica [Merck Art. 7734, 5 g] and the residue purified by SPC eluting with System D (1:3 to 2:5) to give the title compound (1.3 g).

T.l.c. System D (2:5) Rf 0.45.

### Intermediate 50

### 5-Bromo-3H-benzofuran-2-one

A mixture of 5-bromo-2-methoxybenzeneacetonitrile (2.0 g) and sulphuric acid (50%; 20 ml) was refluxed for 16 h, diluted with water (40 ml) and extracted with ethyl acetate (2×100 ml). The dried extract was evaporated to leave the intermediate methoxybenzeneacetic acid as a white solid (1.85 g). The solid in dichloromethane (40 ml) at −78° was treated with boron tribromide (1M in dichloromethane 25 ml) dropwise and allowed to warm to room temperature. The solution was stirred for 2 h and treated dropwise with water (50 ml) (cautiously). The mixture was extracted with dichloromethane (2×50 ml) and the dried extract was evaporated to leave a white solid. The solid was treated with boiling toluene (30 ml), filtered and the toluene phase was evaporated. The residue was purified on a column of silica eluting with System C (4:1) to give the title compound as a beige solid (750 mg) m.p. 147-148° C.

### Intermediate 51

### Methyl 4′-[[3-[3-(dimethylamino)-1-propynyl]-4-methoxybenzoyl]amino][1,1′-biphenyl]-4-carboxylate

A suspension of the hydrochloride salt of Intermediate 1 (3.03 g) in thionyl chloride (7.37 ml) was heated at reflux for 5 min. The solution was evaporated and the residue co-evaporated with toluene (2×20 ml). A mixture of the residue and methyl 4′-amino[1,1′-biphenyl]-4-carboxylate (2.54 g) in DMF (50 ml) and pyridine (50 ml) was heated to 100° under nitrogen. A further quantity of DMF (50 ml) was added and the reaction mixture heated at 100°, under nitrogen for 4 h. The cooled solution was evaporated and treated with aqueous saturated sodium bicarbonate (100 ml). The solid was filtered off, dried in vacuo, and dissolved in refluxing ethanol (3 litres). The insoluble material was filtered off and the filtrated adsorbed onto silica (Merck 9385) and purified by FCC using gradient elution with ethyl acetate:ethanol (9:1) to System E (80:20:1) to give an orange solid (2.8 g). The solid was crystalised from ethanol:THF (2:1) to give the title compound (1.54 g). m.p. 234-237°.

### Intermediate 52

### 2-Methoxyethyl 4-bromo-3-methylbenzoate

4-bromo-3-methylbenzoic acid (10.0 g) was treated with 2-methoxyethanol (100 ml) and concentrated sulphuric acid (7.45 ml) and the solution stirred at 100° C. under nitrogen for 24 h. When cool, the solution was evaporated, brought to pH9 with aqueous sodium bicarbonate (8% w/w, 300 ml) and anhydrous sodium carbonate, and extracted with ethyl acetate (3×300 ml). The dried, combined organic extracts were evaporated, adsorbed onto silica gel (Merck 7734, 60 ml) and purified by FCC eluting with hexane:ethyl acetate:acetic acid (193:5:2) to give a pale brown-yellow oil (8.73 g). This oil was adsorbed onto silica gel (Merck 7734, 55 ml) from hot ethanol (250 ml) and purified by SPC eluting with the same solvent as above to give the title compound as an orange-brown light oil (8.58 g).

T.l.c. hexane ethyl acetate:acetic acid (89:10:1), Rf 0.37.

### Intermediate 53

### 2-Methoxyethyl 4′-[bis(phenylmethyl)amino-2-methyl]-1,1′-biphenyl]-4-carboxylate

A hot mixture of Intermediate 52 (1.72 g) and Intermediate 6 (2.0 g) in DME (50 ml) was added to a solution of anhydrous sodium carbonate (0.94 g) in water (25 ml). Tetrakis (triphenylphosphine)palladium (0) (0.277 g) was added and the mixture stirred at reflux under nitrogen for 6 h. The cool mixture was evaporated, the residue absorbed onto silica gel (Merck 7734, 25 ml) from hot ethanol (250 ml), and purified by FCC eluting with hexane:ethyl acetate (9:1) to give the title compound as a cream/yellow solid (2.57 g) m.p. 91-93°.

### Intermediate 54

### 2-Methoxyethyl 4′-amino-2-methyl[1,1′-biphenyl]-4-carboxylate

A solution of Intermediate 53 (2.45 g) in DMF (80 ml) was added to a pre-hydrogenated mixture of 10% palladium-on-carbon (1.0 g) and DMF (30 ml) and the mixture hydrogenated at room temperature and pressure for 54 h until uptake ceased. The catalyst was filtered off and the filtrate evaporated. The residue was treated with ethanol (100 ml), filtered, and the filtrate adsorbed onto silica gel (Merck 7734, 7 g). Purification by FCC eluting with ethyl acetate:hexane (1:2) gave the title compound as cream-coloured crystals (1.38 g), m.p. 72-75°.

### Intermediate 55

### 2-(Dimethylamino)ethyl 4-bromo-3-methylbenzoate

4-bromo-3-methylbenzoic acid (8 g) was treated with thionyl chloride (12 ml) and stirred at reflux for 2 h. When cool, the solution was evaporated and then co-evaporated with toluene (2×15 ml). The remaining liquid was treated with 2-dimethylaminoethanol (4.1 ml), followed by pyridine (10 ml), under ice-cooling, and then the reaction mixture was allowed to reach room temperature and stirred for a further 2 h. Aqueous sodium bicarbonate (8%, 20 ml) was added and the resultant solution was then evaporated down to a small volume. The solution/slurry was then acidified with hydrochloric acid (2N; 100 ml) and extracted with ethyl acetate (2×100 ml). The organic extracts were discarded and the aqueous component basified with sodium bicarbonate (8%). The resultant aqueous solution was extracted with ethyl acetate (3×250 ml), and the combined, dried extracts were evaporated in vacuo to give the title compound as a slightly impure oil. Purification by FCC eluting with System A (97:3:0.3) afforded the title compound as a pale yellow oil (5.46 g).

| | | | | |
|---|---|---|---|---|
| Analysis Found: | C, 50.3; | H.5.7; | N, 4.9; | Br, 27.7 |
| C₁₂H₁₆BrNO₂ requires | C, 50.4; | H, 5.6; | N, 4.9; | Br, 27.9%. |

### Intermediate 56

### (2,2-Dimethyl-1,3-dioxolan-4-yl)methyl-4-bromo-3-methylbenzoate

A slurry, of 2,2-dimethyl-1,3-diozolane-4-methanol (0.963 ml), 4-bromo-3-methylbenzoic acid (2 g), 4-pyrrolidinopyridine (0.22 g) and 4-[2-[(cyclohexylcarbonimidoyl)amino]ethyl]-4-methylmorpholinum 4-methylbenzenesulphonate (5.5 g) in dry dichloromethane (50 ml) was stirred vigourously at room temperature, under nitrogen, for 72 h. The mixture was evaporated in vacuo to dryness and then sodium bicarbonate was added (8%, 80 nm). The aqueous solution was extracted with ether (3×100 ml), and the combined, dried organic extracts were evaporated onto silica gel (Merck Art 7734). Purification by FCC eluting with System C (9:1) gave the title compound as a yellow oil (1;53 g).

T.l.c. System C (9:1) Rf 0.15

### Intermediate 57

### 2,3-Dihydroxypropyl] 4-bromo-3-methylbenzoate

Intermediate 56 (1.27 g) was dissolved in ThF (50 ml) containing hydrochloric acid (2N; 20 ml) and stirred at room temperature for 80 h. Sodium carbonate (8%, 80 ml) was added and the resultant solution was evaporated in vacuo. The remaining solution was extracted with ethyl acetate (3×150 ml) and the combined, dried extracts were evaporated in vacuo to give an oil. This oil was purified by FCC eluting with System C (15:85) to give the title compound as a colourless oil (0.858 g).

T.l.c. (ether) Rf 0.3.

### Intermediate 58

### (±)2-Methoxy-1-methylethyl 4-bromo-3-methylbenzoate

A mixture of (±)2-methoxy-1-methylethanol (100 ml), 4bromo-3-methylbenzoic acid (10.0 g), and concentrated sulphuric acid (7.45 ml) were stirred at 90-100° under nitrogen for 18 h. The solution was evaporated and the residue was basified to pH8 by the addition of aqueous saturated sodium bicarbonate (300 ml). The mixture was extracted with ether (5×200 ml) and the combined, dried organic extracts were evaporated. The residual oil was purified by FCC, eluting with ether acetate:hexane (1:49) to give the crude title compound (7.67 g). A portion of this (4.0 g) was purified by SPC (Merck 7729) eluting with ethyl acetate:hexane (25:975) to give the title compound as a light orange oil (3.4 g).

| | | | |
|---|---|---|---|
| Analysis Found: | C, 50.3; | H, 5.3; | Br, 27.9 |
| C₁₂H₁₅BrO requires | C, 50.2; | H, 5.3; | Br, 27.8% |

### Intermediate 59

### [4-[[3-[3-(Dimethylamino)propyl]-4-methoxybenzoyl]amino]-3-methylphenyl]boronic acid

A solution of n-butyl lithium (1.68M; 6 ml) in hexane was added dropwise to a solution of Intermediate 45 (1.4 g) in TBF (20 ml) at −78° for 1 h, warmed to 0°, and re-cooled to −78°. Triisopropylborate (1.88 g) was added dropwise and the solution was stirred at −78° for 1 h and at room temperature for 2 h. Water (50 ml) was added and the mixture was extracted with dichloromethane (4×75 ml). The dried extract was evaporated and the residue was purified by FCC eluting with System F (190:10:1) followed by (90:10:1) to give the title compound as a white foam (220 mg).

T.l.c. System F (90:10:1), Rf 0.25

### Intermediate 60

### 2-Methoxyethyl 4-iodo-2-methoxybenzoate

A solution of 4-iodo-2-methoxybenzoic acid (900 mg) in 2-methoxyethanol (10 ml) was treated dropwise with sulphuric acid (18N; 0.1 ml) and heated at 100° for 22 h The solution was basified with aqueous sodium bicarbonate (1M) and evaporated. The residue was partitioned between aqueous sodium bicarbonate (1N; 50 ml) and ethyl acetate (3×50 ml). The dried extracts was evaporated to give an oil which was purified by FCC eluting with System C (4:1) followed by (3:2) to give the title compound as a colourless oil (1.0 g).

T.l.c. System C (1:1), Rf 0.45

### Intermediate 61

### 4-Bromo-N-(methanesulphonyl)-3-methylbenzamide

DMF (5 drops) was added to a solution of 4-bromo-3-methylbenzoic acid (2.0 g) and oxalyl chloride (1.06 g) in dichloromethane (50 ml) under nitrogen. The solution was stirred until effervescence ceased (1 h), and was evaporated. The residue was treated with methanesulphonamide (940 mg) and the mixture was heated at 130° for 3 h. During this time the solid mixture liquified and gradually resolidified to leave an orange solid. The solid was purified by FCC eluting with ether followed by ethyl acetate to give the title compound as a white foam (405 mg).

| | | | |
|---|---|---|---|
| Analysis Found: | C, 32.9; | H, 3.2; | N.4.0 |
| C₉H₁₀BrNO₃S.2H₂O requires | C, 32.9; | H, 4.3; | N, 4.3% |

### Intermediate 62

### 2-Hydroxy-4-iodobenzenemethanol

A solution of borane in THF (1M, 50 ml) was added dropwise to a solution of 2-hydroxy-4-iodobenzoic acid (5.0 g) in THF (50 ml) under nitrogen. The solution was stirred at room temperature for 1 h and at reflux for 1 h. The cooled solution was treated cautiously with hydrochloric acid (2N; 50 ml) and ThF was evaporated in vacuo. The resulting mixture was extracted with dichloromethane (2×100 ml) and the dried extract was evaporated. The residue was crystallised from toluene to give the title compound as a beige solid (2.7 g). m.p. 129-131°

### Intermediate 63

### 7-Iodo-4H-1,3-benzodioxin

A mixture of sodium hydride (80% disp; 350 mg) and DMF (10 ml) under nitrogen was treated slowly with a solution of Intermediate 62 (0.5 g) in dichloromethane (5 ml). The mixture was heated at 80° for 25 min, cooled, and treated dropwise with water (50 ml). The resulting emulsion was extracted with dichloromethane (2×100 ml) and the dried extracts was evaporated to leave an orange oil. The oil was purified by FCC eluting with System C (9:1) to give the title compound as a white solid (125 mg).

T.l.c. System C (3:1) Rf 0.6.

### Intermediate 64

### 4-(Bromomethyl)-4′-nitro-1,1′-biphenyl

To a solution of 4-methyl-4′-nitro[1,1′-biphenyl] (2 g) in carbon tetrachloride (40ml) was added to a trace of benzoyl peroxide and N-bromosuccinimide (1.67 g). The mixture was heated under reflux and irradiated with white light (150 Watts) for 30 min. The mixture was filtered, the residue washed with carbon tetrachloride. (10 ml) and the combined filtrate and washings evaporated to give a yellow solid consisting of the title compound (2.75 g).

T.l.c. cyclohexane:ethyl acetate (15:1) Rf 0.38.

### Intermediate 65

### 4-(1H-Imidazol-1-ylmethyl)-4′-nitro-1,1′-biphenyl

To a solution of imidazole (0.35 g) in dry DMF (10 ml) was added sodium hydride (0.154 g of an 80% dispersion in oil). After 10 min, a solution of Intermediate 64 (1.5 g) in dry DMF (5 ml) was added. The solution was stirred at room temperature for 3.5 h and evaporated to dryness. The residue was mixed with ethyl acetate (200 ml), washed with water (2×50 ml) and extracted with 2N hydrochloric acid (3×100 ml). The combined extracts were washed with ether (2×50 ml) and the pH of the solution adjusted to 11 with 40% aqueous sodium hydroxide. The mixture was extracted with dichloromethane (4×75 ml), the combined extracts dried then filtered and the filtrate evaporated to dryness to give the title compound (0.919 g) as a yellow solid.

### Intermediate 66

### 4′-(1H-Imidazol-1-ylmethyl)-[1,1′-biphenyl]-4-amine

A solution of Intermediate 65 (0.708 g) in acetic acid (5 ml) containing 10% palladium on carbon (0.3 g) was hydrogenated at atmospheric pressure and room temperature for 3 h. The mixture was filtered and the combined filtrate and washings were evaporated to give a yellow crystalline product which was triturated with ether to give the title compound as a cream-coloured solid (0.488 g).

T.l.c. System B (10:1) Rf 0.26

### Intermediate 67

### 3-(4-Bromo-3-methylphenyl)-5-methyl-1,2,4-oxadiazole

A solution of sodium methoxide (1.93 g) in methanol (15 ml) was added dropwise over 10 min to a solution of hydroxylamine hydrochloride (2.48 g) in methanol (30 ml). The mixture was stirred for 1 h at 20° and was then filtered. 4-Bromo-3-methylbenzonitrile (7 g) was then added to the filtrate, and the mixture heated to reflux for 18 h. The solvent was then evaporated giving a grey solid, a portion of which (2.2 g) was dissolved in acetic anhydride (6 ml) and heated to 800 for 18 h. The reaction was cooled and was poured into water (100 ml). The solid was separated, collected and recrystallised from isopropanol (20 ml) giving the title compound as colourless microcrystals (896 mg) m.p. 78°.

### Intermediate 68

### 2′-Methyl-4′-(5-methyl-1,2,4-oxadiazol-3-yl)[1,1′-biphenyl]-4-carboxylic acid

A mixture of 4-carboxyphenylboronic acid (150 mg), Intermediate 67 (228 mg), sodium carbonate (412 mg) and tetrakis(triphenylphosphine)palladium (0) (21 mg) in 1:1 aqueous DME (20 ml) was heated at reflux under nitrogen for 18 h. The mixture was allowed to cool, acidified with 2N HCl and then extracted with ethyl acetate (2×40 ml). The dried extracts were evaporated to give a cream-coloured solid (285 mg). This was recrystallised from isopropanol (5 ml) giving the title compound as a fawn solid (165 mg).

mp. 229-231°

### Intermediate 69

### 5-(4-Bromo-3-methylphenyl)-3-methyl-1,2,4-oxadiazole

Sodium metal (602 mg) was added to a suspension of molecular sieves (4A) in absolute ethanol (30 ml) under nitrogen at 20°. After 15 min N-hydroxyethanimidamide (1.94 g) was added. Stirring was maintained for 1 h whereupon methyl 4-bromo-3-methylbenzoate (1 g) was added. The mixture was heated to reflux for 1.5 h, then filtered and the filtrate evaporated to dryness. The residue was dissolved in water (75 ml) and extracted with ethyl acetate (2×75 ml) and the dried extracts evaporated to give the title compound as a colourless solid (856 mg) m.p. 75-77°.

### EXAMPLE 1

### (a) N-(4′-Acetyl][1,1′-biphenyl]-4-yl)-3-[3-(dimethylamino)propyl]-4-methoxybenzamide

A solution of the free base of the product of Example 4 (158 mg) in dry 1,4-dioxan (9 ml) was treated with activated manganese (IV) oxide (157 mg) and the mixture stirred at reflux for 2 h. The mixture was filtered and the filter cake washed with hot ethanol. The combined filtrate and washings were evaporated and the residue purified by SPC eluting with System A (945:50:5 934:60:6) to afford a solid which was twice crystallised from ethanol to give the title compound (36 mg) as fine white crystals,

T.l.c. (System A 923:70:7) Rf 0.19

| | | | |
|---|---|---|---|
| Analysis Found: | C, 75.2; | H, 7.0; | N, 6.2. |
| C₂₇H₃₀N₂O₃ requires | C, 75.3; | H, 7.0; | N, 6.5%. |

Similarly prepared was:

### (b) N-(4′-Acetyl[1,1′-biphenyl]-4-yl)-3-[2-(dimethylamino)ethyl]-4-methoxybenzamide as a cream-coloured solid (131 mg) m.p. 236-240°

| | | | |
|---|---|---|---|
| Analysis Found: | C, 75.05; | H, 6.8; | N, 6.7; |
| C₂₆H₂₈N₂O₃ requires | C, 75.0; | H, 6.8; | N, 6.7%. |

From the product of Example 8 (281 mg).

### EXAMPLE 2

### 4′-[[3-[3-(Dimethylamino)propyl]-4-methoxybenzoyl]amino][1,1′-biphenyl]-4-carboxylic acid maleate

A mixture of 4-boronobenzoic acid (181 mg) and Intermediate 20 (427 mg) in DME (10 ml) was treated with a solution of sodium carbonate (116 mg) in water (5 ml), followed by tetrakis(triphenylphosphine)palladium (0) (50 mg), and the stirred mixture was heated at reflux under nitrogen for 4 h. The mixture was evaporated, and the residue purified by FCC using gradient elution with System A (78:20:2 to 56:40:4) to afford a white solid (271 mg). A portion of the solid (241 mg) was treated with maleic acid (69 mg) and ethanol (20ml) was added gradually at reflux until the solids dissolved. On cooling, the white solid was collected to give the title compound (143 mg) as fine white crystals, m.p. 189-193°.

| | | | |
|---|---|---|---|
| Analysis Found: | C, 65.7; | H, 5.8; | N, 5.0. |
| C₂₆H₂₈N₂O₄.C₄H₄O₄ requires | C, 65.7; | H, 5.9; | N, 5.1%. |

Similarly prepared was:

### EXAMPLE 3

### 3-[3-(Dimethylamino)propyl]-N-(4′-formyl-2′-methyl[1,1′-biphenyl]-4-yl)-4-methoxybenzanide, ethanedioate (1:1)

(314 mg) as a colourless, crystalline solid, m.p. 177.5-180.5°.

| | | | |
|---|---|---|---|
| Analysis Found: | C, 66.85; | H, 6.2; | N, 5.3. |
| C₂₇H₃₀N₂O₃.C₂H₂O₄ requires | C, 66.9; | H, 6.2; | N, 5.4%. |

From Intermediate 20 (1.174 g) and Intermediate 28 (514 mg). After 7 h, the reaction mixture was cooled, diluted with 2N aqueous sodium carbonate (100 ml), and extracted with dichloromethane (3×100 ml). The combined extracts were dried, evaporated and purified by SPC using System A (95:5:0.5) followed by (90:10:0.5) as eluent, to give the free base of the title compound (1.25 g) as a viscous gum, which still contained some ethanol. T.l.c. (System A 95:5:0.5) Rf 0.15. A sample (314 mg) of this material in ethyl acetate (15 ml) was added slowly to a boiling solution of anhydrous oxalic acid (74 mg) in ethyl acetate (35 ml). After cooling, the solid was collected, washed with ethyl acetate, and dried under high vacuum at 70°, to give the title compound.

### EXAMPLE 4

### 3-[3-(Dimethylamino)propyl]-N-[4′-(1-hydroxyethyl)[1,1′-biphenyl]-4-yl) 4-methoxybenzamide hydrochloride

A mixture of Intermediate 2 (300 mg) in thionyl chloride (0.7 ml) with a catalytic quantity of dry DMF (1 drop) was heated at reflux for 15 min. The reaction mixture was cooled and the thionyl chloride was evaporated. The residue was re-evaporated with dry toluene (2×7 ml) to leave a buff coloured solid. A solution of the solid and Intermediate 13 (270 mg) in dry pyridine (5 ml) was stirred at room temperature for 5 h. The reaction mixture was then added to aqueous sodium carbonate (2M; 25 ml) and extracted with dichloromethane-isopropanol (9:1, 4×20 ml). The combined extracts were dried, filtered, and evaporated to give a solid. The solid was adsorbed on silica and purified by FCC eluting with System A (150:8:1) to give recovered 4′-amino-α-methyl-[1,1-biphenyl]-4-methanol (49 mg) and then with System A (100:8:1) to give a white solid (385 mg). A solution of the solid in ethyl acetate:methanol (5:1, 60 ml) was treated with excess ethereal hydrogen chloride. The resultant solid was filtered off, washed with dry ether, and dried under vacuum to give the title compound (280 mg) as an off-white crystalline solid, m.p. 242-244°.

T.l.c. System A (50:8:1) Rf 0.28.

Similarly prepared were:

### EXAMPLE 5

### 3-[3-(Dimethylamino)propyl]-N-(2′-(1-hydroxyethyl)[1,1′-biphenyl]-4-yl]-4-methoxybenzamide hydrochloride

(455 mg) as a white crystalline solid m.p. 224-227°.

T.l.c. System E (85:15:1) Rf 0.14.

From Intermediate 2 (400 mg) and Intermediate 9 (360 mg) with a reaction time of 36 h. Extraction was carried out using dichloromethane and purification was by FCC eluting with System E (85:15:1).

### EXAMPLE 6

### 3-[3-(Dimethylamino)propyl]-N-(4′-ethyl[1,1′-biphenyl]-4-yl)-4-methoxybenzamide

(487 mg) as white crystalline needles, m.p. 200-202°.

T.l.c. System A (100:8:1) Rf 0.25.

From Intermediate 2 (529 mg) and Intermediate 14(a) (440 mg) with a reaction time of 72 h. Extraction was carried out using chloroform and purification was by FCC eluting with System E (90:10:1) to give a white crystalline solid. The solid was further purified by crystallisation from isopropyl acetate (40 ml) to give the title compound as a free base.

### EXAMPLE 7

### 4′-[[3-[3-(Dimethylamino)propyl]-4-hydroxybenzoyl]amino][1,1′-biphenyl]-4-carboxylic acid

A hot solution of 4-boronobenzoic acid (678 mg) and Intermediate 21 (1.54 mg) in DME (70 ml) was treated with a solution of sodium carbonate (692 mg) in water (35 ml), followed by tetrakis(triphenylphosphine)palladium (0) (189 mg) and stirred under nitrogen at reflux for 3 h. The mixture was evaporated and purified by FCC eluting with System A (67:30:3 to 50:45:5) to afford a solid. The solid was triturated with ethanol (40 ml) and the precipitate filtered off to give the title compound (837 mg) as fine white crystals, m.p. 278-280°.

| | | | |
|---|---|---|---|
| Analysis Found: | C, 70:8; | H, 6.0: | N, 6.35 |
| C₂₅H₂₆N₂O₄.0.35 H₂O requires: | C, 70.7; | H, 6.3; | N, 6.6% |

| | |
|---|---|
| Water assay Found: | 1.47% w/w H₂O = 0.35 mole. |

### EXAMPLE 8

### 3-[2-Dimethylamino)ethyl]-N-[4′-(1-hydroxethyl)[1,1′-biphenyl]-4-yl]-4-methoxybenzamide

A suspension of the free base of Intermediate 19 (945 mg) in thionyl chloride (7 ml) was treated with dry DMF (1 drop) and heated at reflux for 1 h. The solution was evaporated and the residue co-evaporated with toluene (4×10 ml). The residue was treated with Intermediate 13 (903 mg), followed by dry pyridine (10 ml), and heated at 80-100° for 1 h under nitrogen. The cooled solution was evaporated and treated with aqueous saturated sodium bicarbonate (50 ml). The solid was filtered off and air-dried for 30 min to give solid (I). The filtrate was extracted with ethyl acetate (7×150 ml), and the combined, dried organic extracts were evaporated. The residue was mixed with solid (I) and adsorbed from hot ethanol onto silica gel (Merck 7734). The resultant silica gel was applied as a plug to a flash column and gradient elution with System A (945:50:5 to 934:60:6) afforded a solid (340 mg). A portion of this solid (80 mg) was crystallised from ethanol to give the title compound (30 mg) as a cream-coloured solid.

T.l.c. System A (89:10:1) Rf 0.19.

| | | | |
|---|---|---|---|
| Analysis Found: | C, 74.35; | H, 7.2; | N, 6.55 |
| C₂₆H₃₀N₂O₃ requires | C, 74.6; | H, 7.2; | N, 6.7%. |

### EXAMPLE 9

### 4′-[[3-[3-(Dimethylamino)propyl]-4-methoxybenzoyl]amino]-N,N-dimethyl[1,1-biphenyl]-4-carboxamide

A solution of Intermediate 2 (1.74 g) in dry ThF (40 ml) and dry DMF (40 ml) was treated under nitrogen with triethylamine (2.05 ml), followed at −10 to −5° by methanesulphonyl chloride (0.57 ml). After 1.5 h, Intermediate 11 (1.885 g) was added and after 2 h the mixture was stirred at room temperature overnight, then heated at reflux for 1 h. The mixture was evaporated to give a yellow solid, sodium bicarbonate (8%, 70 ml) was added and the mixture extracted with ethyl acetate (4×50 ml). The combined, dried organic extracts were evaporated to give yellow crystals which on recrystallisation from ethyl acetate gave cream crystals (1.2 g). The crystals were recrystallised from ethyl acetate (70 ml) to give a cream powder which was dried under vacuum at 50° to give the title compound (0.79 g), m.p. 180-182°.

T.l.c. System A (824:160:16) Rf 0.56.

### EXAMPLE 10

### 4′-[[3-[3-(Dimethylamino)propyl]-4-methoxybenzoylamino][1,1′-biphenyl]-4-carboxamide maleate

A solution of Intermediate 2 (670 mg) in dry THF (15 ml) and dry DMF (15 ml) was treated under nitrogen with triethylamine (0.79 ml) and followed at −15 to −10° by methanesulphonyl chloride (0.22 ml). After 2 h, Intermediate 12 (600 mg) was added, and after 2 h, the mixture was stirred at 23° for 3 days. The mixture was evaporated and the residue treated with aqueous saturated sodium bicarbonate (35 ml). The precipitate was filtered off, air-dried for 30 min, and then dried in vacuo over phosphorus pentoxide for 1 h. The solid was adsorbed from ethanol onto silica gel (Merck 7734, 15 g) and the resultant silica gel applied as a plug to a flash column. Elution with System A (945:50:5 to 890:100:10) afforded firstly recovered 4′-amino[1,1′-biphenyl]-4-carboxamide and secondly the free base of the title compound (412 mg) as a white solid. T.l.c. System A 89:10:1) Rf 0.09. A mixture of the free base (376 mg) and maleic acid (121 mg) was dissolved in hot ethanol (130 ml), filtered, and on cooling the title compound (294 mg) crystallised as fine white crystals.

| | | | |
|---|---|---|---|
| Analysis Found: | C, 65.7; | H, 6.1; | N, 7.5. |
| C₂₆H₂₉N₃O₃.C₄H₄O₄ requires | C, 65.8; | H, 6.1; | N, 7.7%. |

n.m.r. (DMSO-d₆) δ 1.94 (2H,m), 2.68 (2H,t), 2.78 (6H,s), 3.09(2H,m), 3.92 (3H,s), 6.05 (2H,s), 7.43+8.06 (2H, 2×br.s), 7.17+7.75-8.0 (11H,m), 10.25 (1H,s).

Similarly prepared was:

### EXAMPLE 11

### N-[4′-(Aminosulphonyl)[1,1′-biphenyl]-yl]-3-[3-(dimethylamino)propyl]-4-methoxybenzamide maleate

(140 mg) as cream-coloured crystals, m.p. 226-228°,

| | | | |
|---|---|---|---|
| Analysis found: | C, 59.7; | H, 5.7; | N, 7.1. |
| C₂₅H₂₉N₃O₄S.C₄H₄O₄ requires | C, 59.7; | H, 5.7; | N, 7.2% |

From Intermediate 2 (0.566 mg) and 4′-Amino[1,1′-biphenyl]-4-sulphonamide (0.587 mg). Except that on evaporation of the reaction mixture the residue was adsorbed from refluxing methanol onto silica gel (Merck 7734) and purified by SPC eluting with System A (945:50:5 to 89:10:1).

### EXAMPLE 12

### 3-[3-(Dimethylamino)propyl]-N-(4′-nitro[1,1′-biphenyl]-4-yl)-4-methoxybenzamide hydrochloride

A stirred suspension of Intermediate 2 (500 mg) in dry THF (20 ml) and dry DMF (20 ml) was treated under nitrogen at 23° with triethylamine (584 mg) and followed at −5° to −10° by the addition of methanesulphonyl chloride (0.16 mol). After 2 h, 4′-nitro[1,1′-biphenyl]-4-amine (384 mg) was added and after 2 h the mixture was stirred at 23° for 24 h. The mixture was heated at reflux for 16 h, cooled, and evaporated. The residue was treated with aqueous saturated sodium bicarbonate (80 ml), extracted with ethyl acetate (3×80 ml), and the combined, dried organic extracts were evaporated. The residue was purified by FCC eluting with System A (945:50:5 to 912:80:8) to afford a solid a solution of ethanol (20 ml) was acidified with ethanolic hydrogen chloride to pH1 and the precipitate collected to give the title compound (168 mg) as fine yellow crystals.

T.l.c. System A (945:50:5) Rf 0.22.

| | | | |
|---|---|---|---|
| Analysis Found: | C, 64.0; | H, 6.0; | N, 9.0. |
| C₂₇H₂₇N₃O₄.HCl.0.08C₂H₅OH requires | C, 63.8; | H, 6.1; | N, 8.9%. |

### EXAMPLE 13

### 3-[3-(Dimethylaminopropyl]-N-[4′-fluoro[1,1′-biphenyl]-4-yl]-4-methoxybenzamide maleate

A mixture of the free base of Intermediate 5 (1.0 g), 4-fluoro[1,1′-biphenyl]-4-amine (0.94 g), tri-n-butylamine (2.4 ml) and dichlorobis(triphenylphosphine)palladium (II) (0.20 g) in DMF (2 ml) was heated at 120°, with stirring, under carbon monoxide for 18 h. The mixture was poured into aqueous 8% sodium bicarbonate (50 ml), extracted with ethyl acetate (2×40 ml), and the combined, dried organic extracts were evaporated. The residual yellow-brown solid was purified by SPC eluting with System A (945:50:5) to yield a cream solid (0.46 g). This solid was recrystallised from ethanol (10 ml) to give the free base of the title compound (0.28 g) as white crystals. This was dissolved in hot ethanol and maleic acid (85 mg) in warm ethanol (2 ml) was added. The resultant solution was evaporated and the white solid residue was recrystallised from ethanol (2 ml) to give the title compound (0.27 g) as white crystals, m.p. 165-166°.

| | | | |
|---|---|---|---|
| Analysis found | C, 66.5; | H, 5.9; | N, 5.2. |
| C₂₃H₂₇FN₂O₂.C₄H₄O₄ requires | C, 66.7; | H, 6.0; | N, 5.4% |

Similarly prepared were:

### EXAMPLE 14

### (a) 3-[3-(Dimethylaminopropyl]-N-[4′-[(dimethylamino)sulphonyl][1,1′-biphenyl]-4-yl-4-methoxybenzamide maleate

(61 mg), m.p. 140-141.5°,

| | | | |
|---|---|---|---|
| Analysis Found: | C, 60.9; | H, 6.1; | N, 6.75. |
| C₂₇H₃₃N₃O₄S.C₄H₄O₄ requires | C, 60.9; | H, 6.1; | N, 6.9%. |

From the free base of Intermediate 5 (868 mg) and Intermediate 15 (750 mg) with purification by SPC using gradient (967:30:3 to 934:60:6). Elution with System A initially formed the free base of the title compound (465 mg) which was used to prepare the title compound.

### (b) 3-[3-(Dimethylamino)propyl]-4-methoxy-N-[4′-methylsulphonyl)[1,1′-biphenyl]-4-yl]benzamide maleate

(316 mg), m.p. 173-177.5°.

| | | | | |
|---|---|---|---|---|
| Analysis Found: | C, 62.0; | H, 6.0; | N, 4.6; | S, 5.3. |
| C₂₆H₃₀N₂O₄S.C₄H₄O₄ requires | C, 61.8; | H, 5.9; | N, 4.8; | S, 5.5%. |

From the free base of Intermediate 5 (800 mg) and 4′-(methylsulphonyl)[1,1′-biphenyl]-4-amine (618 mg) with purification by SPC using gradient elution with System A (945:50:5 to 934:60:6). A portion (421 mg) of the initially formed impure free base of the title compound (437 mg) was used to prepare the title compound.

### (c) N-(4′-Cyano[1,1′-biphenyl]-4-yl)-3-[3-(dimethylamino)propyl]-methoxybenzamide maleate

(44 mg), m.p. 200-202.5°.

| | | | |
|---|---|---|---|
| Analysis Found: | C, 68.2; | H, 6.1; | N, 7.9. |
| C₂₆H₂₇N₃O₂.C₄H₄O₄ requires | C, 68.0; | H, 5.9; | N, 7.9%. |

From 4′-amino-[1,1′-biphenyl]-4-carbonitrile (493 mg) and the free base of Intermediate 5 (812 mg) with purification by SPC eluting with System A (956:40:4 to 934:60:6). The initially formed impure free base of the title compound (145 mg) was used to prepared the title compound.

### EXAMPLE 15

### 4′-[[3-[3-(Dimethylamino)propyl]-4-methoxybenzoyl]amino][1,1′-biphenyl]-3-carboxamide hydrochloride

(185 mg) T.l.c. System A (12:1:87) Rf 0.11,

| | | | |
|---|---|---|---|
| Analysis Found: | C, 65.7; | H, 6.6; | N, 8.6. |
| C₂₆H₂₉N₃O₃.HCl.0.33H₂O.0.33C₂H₅OH | C, 65.5; | H, 6,7; | N, 8.6%. |
| requires | | | |

Water assay found 1.23% H₂O=0.33 mol H₂O

From Intermediate 16(a) (600 mg) and the free base of Intermediate 5 (902 mg) with purification by SPC using gradient elution with System A (923:70:7 907:85:8). The initially formed free base of the title compound (493 mg) was dissolved in hot ethanol (35 ml), treated with ethanolic hydrogen chloride (2 ml) and cooled to give the title compound.

### EXAMPLE 16

### 3′-[[3-[3-(Dimethylamino)propyl-4-methoxybenzoyl]amino][1,1′-biphenyl]-4-carboxamide hydrochloride

(115 mg), m.p. 145°.

| | | | |
|---|---|---|---|
| Analysis Found: | C, 64.7; | H, 6.8; | N, 8.2. |
| C₂₆H₂₉N₃O₃.HCl.0.3 CH₃CO₂C₂H₅ | C, 64.8; | H, 6.5; | N, 8.3%. |
| requires | | | |

From Intermediate 7 (1.56 g) and the free base of Intermediate 5 (2.35 g) with purification by FCC eluting with System A (89:10:1). Two fractions of the impure free base of the title compound were initially formed. The initial fractions was further purified by SPC eluting with System A (89:10:1) to give a pale cream solid which was crystallised from ethyl acetate. A hot solution of the free base in refluxing ethyl acetate was added to ethereal hydrogen chloride (3 ml) and the precipitate was filtered off to give the title compound.

### EXAMPLE 17

### 4′-[[3-[3-(Dimethylamino)propyl-4-hydroxybenzoyl]amino]-N,N-dimethyl[1,1′-biphenyl]-4-carboxamide

Sodium hydride (0.470 g, dispersion in oil) was washed under nitrogen with hexane, treated with dry DMF (6 ml) and then ethanethiol (1.06 ml) in dry DMF (6 ml) was added dropwise over 5 min. After 1 h, DMF (2 ml) was added, followed by a solution of the product of Example 9 (0.658 g) in dry DMF (8 ml). The solution was heated at 1500 under nitrogen for 18 h and then at 2000 for 2 h. The cooled mixture was acidified to pH1 with 2N hydrochloric acid and basified to pH8 with saturated sodium bicarbonate. The mixture was extracted into ethyl acetate (20×100 ml) and the combined dried organic extracts were evaporated. The residue (500 mg) was adsorbed from refluxing methanol (500 ml) onto silica gel (Merck 9385) and this was purifed by FCC eluting with System A (18.4:1.4:0.14 to 17.5:2.5:0.75) to give the title compound (296 mg) as a white power, m.p. 235.0-237.5°.

T.l.c. System A (175:2:5:0.25) Rf 0.48.

### EXAMPLE 18

### N-(4′-Cyano[1,1′-biphenyl]-4-yl)-3-[3-(dimethylamino)propyl]-4-methoxybenzamide hydrochloride

The combined mother liquors obtained from recrystallisation of the product of Example 14(c) were evaporated and then treated with aqueous saturated sodium bicarbonate (50 ml). The mixture was extracted with ethyl acetate (4×80 ml) and the combined, dried organic extracts were evaporated. The residue in ethanol (2 ml) was acidified to pH2 with ethanolic hydrogen chloride and on cooling, the precipitate was collected to give the title compound (27 mg) as fine white crystals, m.p. 222-225.5°.

| | | | |
|---|---|---|---|
| Analysis Found: | C, 69.4; | H, 6.3; | N, 9.2. |
| C₂₆H₂₇N₃O₂.HCl requires: | C, 69.4; | H, 6.3; | N, 9.3% |

### EXAMPLE 19

### 4′-[[[3-[(Dimethylamino)propyl]-4-methoxyphenyl]methyl]amino]-α-methyl1,1′-biphenyl]-4-methanol

A cooled (ice-bath) suspension of lithium aluminium hydride (0.493 g) in dry THF (10 ml) was treated dropwise with a solution of the free base of the product of Example 4 (1.88 g) in dry THF (50 ml). The reaction mixture was then heated at reflux for 1.5 h. The reaction mixture was carefully treated with hydrochloric acid (2M; 75 ml) and then basified with aqueous sodium hydroxide (5M; 35 ml). The aqueous phase was extracted with chloroform (3×100 ml). The combined extracts were dried, filtered and evaporated to give a light brown gum which was purified by FCC eluting with System E (95:5:1) to give the title compound (1.431 g) as an off-white solid, m.p. 148-152°.

| | | | |
|---|---|---|---|
| Analysis Found | C, 77.2; | H, 8.4; | N, 6.5; |
| C₂₇H₃₄N₂O₂ requires | C, 77.5; | H, 8.2; | N, 6.7% |

### EXAMPLE 20

### (a) 3-[3-(Dimethylamino)propyl]-4-methoxy-N-(4′-methoxy[1,1′-biphenyl]-4-yl)benzamide

A stirred mixture of the hydrochloride salt of Intermediate 2 (1.095 g) in thionyl chloride (10 ml) was heated at 90-100° until gas evolution ceased (20 min). The solution was cooled and evaporated to dryness to give a solid. A solution of this solid in dry dichloromethane (20 ml) was diluted with dry pyridine (20 ml) and treated with Intermediate 29 (731 mg). The resulting suspension was stirred at room temperature for 17 h and then heated at reflux for 3 h. Water (5 ml) was added, with vigorous stirring for 30 min. After addition of 2N aqueous sodium carbonate (5 ml), the mixture was evaporated to dryness and then purified by FCC eluting with System F (95:5:0.5) to give the major product (700 mg) as a cream coloured solid. Crystallisation from toluene gave the title compound (623 mg) as a cream coloured crystalline solid, m.p. 198.5-200.5°.

T.l.c. System F (95:5:0.5) Rf 0.26.

Similarly prepared were:

### (b) 3-[3-(Dimethylamino)propyl]-N-[3′-hydroxy-4′-(hydroxymethyl)[1,1′-biphenyl]-4-yl]-4-methoxybenzamide

(168 mg) as a cream crystalline solid, m.p. 171-173°.

| | | | |
|---|---|---|---|
| Analysis Found: | C, 71.0; | H, 6.95; | N, 6.1; |
| C₂₆H₃₀N₂O₄.0.2 (CH₃CO₂C₂H₅) requires | C, 71.2; | H, 7.0; | N, 6.2% |

From the hydrochloride salt of Intermediate 2 (369 mg) and Intermediate 22 (279 mg) using DMF as reaction solvent. Purification by SPC eluting with System A (90:10:1) afforded the title compound (274 mg) as a glass which was recrystallised from ethyl acetate.

### (c) 4′-[[3-[3-(Dimethylamino)propyl]-4-methoxybenzoyl]amino]-3-hydroxy(1,1′-biphenyl]-carboxylic acid

(520 mg).

T.l.c. System A (75:25:2.5) Rf 0.19.

| | | | |
|---|---|---|---|
| Analysis Found: | C, 67.25; | H, 6.75; | N, 5.65. |
| C₂₆H₂₈N₂O₅.(C₂H₅OH).0.5H₂O | C, 66.8; | H, 7.0; | N, 5.55%. |
| requires | | | |

Water Analysis Found: 1.84% H₂O w/w=0.5 H₂O

From the hydrochloride salt of Intermediate 2 (441 mg) and Intermediate 10 (345 mg). The reaction mixture was heated in an oil bath (75) for 20 h and then cooled and treated with water (1 ml). After vigorous stirring for 10 min, the mixture was basified to pH12 by addition of 2N aqueous sodium hydroxide (4 ml) and then evaporated to dryness. The residue was purified by SPC (Merck 7729) using System A (75:25:2.5) as eluent, to give the product (573 mg) as a light brown solid. Trituration with hot absolute ethanol (10 ml) gave, after cooling, washing, and collection, the title compound as a pale brown solid.

### EXAMPLE 21

### N-[3-[3-(Dimethylamino)propyl]-4-methoxyphenyl][1,1′-biphenyl]-4,4′-dicarboxamide methanesulphonate

Thionyl chloride (0.14 ml) was added dropwise under nitrogen to a stirred suspension of Intermediate 8(i) (396 mg) in pyridine (5 ml). After 1 h at 23°, the solution was treated at 0° with a solution of Intermediate 27 (376 mg) in pyridine (1 ml) and then stirred at 23° for 20 h. The mixture was treated with aqueous saturated sodium bicarbonate (40 ml) and ethyl acetate (50 ml), then filtered and the residue was dried in vacuo over phosphorus pentoxide. The organic layer was separated and the aqueous further extracted with ethyl acetate (4×100 ml). The combined, dried organic layers were evaporated, treated with the above dried solid, and adsorbed from ethanol onto silica gel. The residue was purified by SPC and gradient elution with System A (89:10:1 to 87:12:1) afforded the free base of the title compound (167 mg). A solution of the free base (167 mg) in hot ethanol (180 ml) was treated with a solution of orthophosphoric acid (76 mg) in ethanol (4 ml), and on cooling the free base of the title compound crystallised (117 mg). A solution of the free base (117 mg) in hot ethanol (1 litre) was treated with a solution of methanesulphonic acid (47 mg) in ethanol (2 ml) and on cooling the title compound (51 mg) crystallised as cream crystals.

T.l.c. System A (89:10:1) Rf 0.06.

| | | | |
|---|---|---|---|
| Analysis Found: | C, 60.2; | H, 6.3; | N, 7.7. |
| C₂₆H₂₉N₃O₃.MeSO₃H.0.52H₂O requires | C, 60.4; | H, 6.4; | N, 7.8% |

Water assay Found: H₂O, 1.75=0.52 mol H₂O.

### EXAMPLE 22

### 3-[3-(Dimethylamino)propyl]-4-hydroxy-N-(4′-hydroxy(1,1′-biphenyl]-4-yl)benzamide

A stirred mixture of Intermediate 21 (919 mg), Intermediate 23 (713 mg), tetrakis(triphenylphosphine)palladium (0) (138 mg), and anhydrous sodium carbonate (318 mg), in water (6 ml) and DME (6 ml) under nitrogen was heated at reflux. After 7.5 h, the solution was cooled, basified with 2N aqueous sodium hydroxide (9 ml), and absorbed onto silica gel (10 g). SPC (Merck 7729) using System A (90:10:1) as eluent gave a product (900 mg) as a colourless glass. (T.l.c. System A (90:10:1) single spot Rf 0.17) This material was combined with that (130 mg) from a previous experiment and dissolved in boiling ethyl acetate (90 ml). The clear supernatant was added gradually to a boiling solution of maleic acid (337 mg) in ethyl acetate (60 ml). After cooling, the product was collected, washed with ethyl acetate, and dried at 70°/0.4 torr to give the maleate of the title compound (1.242 g) as a colourless crystalline solid, m.p. 202-204°. The title compound was recovered from the maleate by basifying its aqueous solution with 8% aqueous sodium bicarbonate (50 ml) and extracting into ethyl acetate:isopropanol (9:1) (4×200 ml). The combined extracts were dried and evaporated to give a colourless gum. Crystallisation from ethyl acetate:hexane gave the title compound (683 mg) as a colourless crystalline solid.

| | | | |
|---|---|---|---|
| Analysis Found: | C, 73.25; | H.6.85; | N.6.85. |
| C₂₄H₂₆N₂O₃.0.07 H₂O requires | C, 73.5; | H, 6.75; | N, 7.15% |

Water Analysis indicates 0.07 moles H₂O

n.m.r. (DMSO-d₆) δ 1.77 (2H,m), 2.20 (6H,s), 2.27 (2H,t), 2.63 (2H,t), 6.8-7.9 (11H), 10.00 (1H,s).

### EXAMPLE 23

### N-[4′-(1-Hydroxyethyl)[1,1′-biphenyl]-4-yl]-3-[3-(methylamino)propyl]-4-methoxybenzamide

A mixture of Intermediate 16(b) (221 mg) in thionyl chloride (1.5 ml) was heated at reflux for 15 min. The thionyl chloride was evaporated and the residue was reevaporated with dry toluene (2×10 ml) to leave an off-white solid (227 mg). A solution of the solid (227 mg) in dichloromethane (6 ml) and dry DMF (1 ml) was added to a solution of Intermediate 13 (182 mg) in dry pyridine (5 ml). The reaction mixture was then stirred at room temperature for 72 h. The reaction mixture was added to aqueous sodium carbonate (2N; 40 ml), extracted with dichloromethane (4×25 ml) and the combined extracts were dried, filtered and the filtrate was purified by FCC. Elution with System A (100:8:1) gave an off-white solid (152 mg). The solid was further purified by crystallisation from ethyl acetate (25 ml) to give the title compound (79 mg) as an off-white crystalline solid m.p. 175-178°.

T.l.c. System A (50:8:1) Rf 0.18.

### EXAMPLE 24

### 3-[3-(Dimethylamino)propyl]-N-[4′-(hydroxymethyl)[1,1′-biphenyl]-4-yl]-4-methoxybenzamide maleate

A mixture of 4-(hydroxymethyl)benzeneboronic acid (240 mg) and Intermediate 20 (618 mg) in DME (15 ml) were treated with a solution of sodium carbonate (176 mg) in water (9 ml), followed by tetrakis(triphenylphosphine)palladium (0) (73 mg), and stirred at reflux under nitrogen for 5 h. When cool, the mixture was evaporated, then treated with water (100 ml), and extracted with ethyl acetate (5×100 ml). The combined, dried organic extracts were evaporated, and the residue purified by FCC eluting with System A (945:50:5 to 923:70:7) to give the free base (545 mg). A portion of the free base (200 mg) was treated with maleic acid (72 mg) followed by ethanol (8 ml) and heated to reflux to effect solution. On cooling the impure title compound (149 mg) crystallised. A portion (100 mg) of this was crystallised from ethanol (3 ml) to give the pure title compound (49 mg) as fine white needles, m.p. 178-180°.

T.l.c. System A (89:10:1) Rf 0.14.

### EXAMPLE 25

### N-[[4′-[3-[3-(Dimethylamino)propyl]-4-methoxybenzoyl]amino]1,1′-biphenyl]-4-yl]carbonyl]glycine ethyl ester maleate (1:1)

To Intermediate 2 (0.52 g) was added thionyl chloride (3 ml) and the mixture heated on a steam bath for 5 min. The excess thionyl chloride was evaporated in vacuo and the solid residue re-evaporated with toluene (10 ml). The solid was suspended in pyridine (5 ml) and the free base of Intermediate 25 (0.597 g) added. The mixture was heated on a steam bath for 20 min, the pyridine evaporated in vacuo and the residue diluted with water (20 ml). Sodium carbonate anhyd. (2 g) was added and the solid residue filtered and washed with water. The solid was dissolved in methanol and the solution evaporated to dryness. The residue was redissolved in methanol (70 ml), the solution filtered and the filtrate evaporated to about 25 ml. The solid which separated was filtered, washed with methanol and dried to give the free base of the title compound (0.678 g) m.p. 212-213° dec. The methanolic filtrate was evaporated to dryness, the residue dissolved in ethanol (10 ml) and a solution of maleic acid (0.4 g) in ethanol (3 ml) added. On cooling, the solid which separated was filtered, washed with ethanol and dried to give the title compound (0.147 g) m.p. 178-180°.

T.l.c. System A (150:8:1) Rf 0.14.

### EXAMPLE 26

### N-[[4′-[[3-[3-(Dimethylaminopropyl-4-methoxybenzoyl]amino][1,1′-biphenyl]-4-yl]carbonyl]glycine hydrochloride

A solution of the free base of the product obtained in Example 25 (0.253 g) and potassium hydroxide (0.53 g of 85%) in ethanol (15 ml) was heated under reflux for 1 h. The ethanol was evaporated in vacuo, water (20 ml) added and the solution re-evaporated to remove residual ethanol. To the alkaline solution was added 5N hydrochloric acid (1.6 ml) and the neutral suspension filtered. The residue was washed with water until free of chloride then with ethanol and ether and dried to give the title compound (0.245 g) m.p. 293-295° dec.

T.l.c. (chloroform:methanol:acetic acid:water 15:5:1:1) Rf 0.32.

### EXAMPLE 27

### 3-[3-(Dimethylaminopropyl]-4-methoxy-N-[4′-(methylsulphinyl)[1.1′-biphenyl]-4-yl]benzamide

A stirred mixture of Intermediate 20 (587 mg), Intermediate 17 (331 mg), tetrakis(triphenylphosphine)palladium (0) (92 mg), and anhydrous sodium carbonate (191 mg), in water (4 ml) and DME (4 ml) under nitrogen was heated under reflux. After 7.5 h, the reaction mixture was cooled, diluted with 2N aqueous sodium carbonate (100 ml), and extracted with dichloromethane (3×70 ml). The combined extracts were dried and evaporated to dryness to give a solid which was purified by SPC (Merck 7729) using System A (90:10:1) as eluent, to give the major product (540 mg) as a fawn coloured solid. Crystallisation from ethyl acetate (23 ml) gave the title compound (476 mg) as a cream coloured crystalline solid, m.p. 189.5-191.5°.

T.l.c. System A (90:10:1) Rf 0.22.

### EXAMPLE 28

### 3-[3-(Dimethylamino)propyl]-N-(4′-hydroxy[1,1′-biphenyl]-4-yl)-4-methoxybenzamide

A catalytic quantity of tetrakis(triphenylphosphine)palladium (0) (84 mg) was added to a degassed mixture of Intermediate 20 (550 mg), Intermediate 23 (386 mg) and anhydrous sodium carbonate (162 mg) in DME (10 ml) and water (5 ml). The reaction mixture was heated at reflux for 6.5 h, and then allowed to stand at room temperature for 60 h, under nitrogen. The reaction mixture was basified with aqueous sodium hydroxide (2N) and purified by FCC (Sorbsil C60) eluting with System A (75:8:1) to give an off-white solid (448 mg). The solid was further purified by crystallisation from isopropanol to give the title compound (319 mg) as a white crystalline solid map. 229-230° C.

T.l.c. (System A 50:8:1) Rf 0.24.

### EXAMPLE 29

### N-(2′-Acetyl[1,1′-biphenyl]-yl)-3-[3-(dimethylamino)propyl]-4-methoxybenzamide, (E)-2-butenedioate (1:1)

A cooled solution of the free base of the product obtained in Example 5 (900 mg) in acetone (25 ml) was treated dropwise with a solution of chromic acid (chromium trioxide; 300 mg in water 1.12 ml and conc. sulphuric acid 0.27 ml), at 0° C. The ice bath was removed and the reaction was allowed to warm to room temperature over a 45 min period. The reaction mixture was basified with aqueous sodium bicarbonate (1M; 30 ml) and extracted with dichloromethane (4×50 ml). The combined extracts were dried, filtered and evaporated to give a gum (1.1 g) which was purified by FCC eluting with System A (150:8:1) to give a colourless gum (700 mg). A solution of the gum (700 mg) in ethyl acetate (30 ml) was treated with a hot solution of fumaric acid (189 mg) in methanol (2 ml) and the mixture was diluted with ether (50 ml). The resulting solid was filtered, washed with dry ether, and dried under vacuum to give the title compound (753 mg) as a white crystalline solid.

T.l.c. System A (100:8:1) Rf 0.24;

| | | | |
|---|---|---|---|
| Analysis Found: | C, 67.4; | H, 6.2: | N, 4.9. |
| C₂₇H₃₀N₂O₃.C₄H₄O₄ requires | C, 67.5; | H, 6.3; | N, 5.1%. |

Water Analysis indicates 0.3H₂O.

### EXAMPLE 30

### 4-[[3-[3-(mimethylamino)propyl]-4-methoxybenzoyl]amino]-2-methyl1,1′-biphenyl]-4-carboxylic acid

A stirred mixture of Intermediate 30 (0.718 g), 4-bromo-3-methylbenzoic acid (0.432 g), tetrakis(triphenylphosphine)palladium (0) (0.116 g) and sodium carbonate (0.852 g) in water (10 ml) and DME (20 ml), was heated at reflux under nitrogen. After 18 h, the reaction mixture was cooled and the reaction contents adsorbed onto silica gel (Merck Art, 7734). Purification by SPC (Merck Art. 7729) eluting with System A (73:27:2.7) gave an impure sample of the title compound which was re-columned, as before, to give pure title compound (0.489 g) as an off-white solid m.p. 145-147° C.

| | | | | |
|---|---|---|---|---|
| Analysis Found: | C, 67.3; | H, 6.9: | N, 6.2; | Cl, 2.4, |
| C₂₇H₃₀N₂O₄.H₂O.0.35 HCl.0.07 CH₃CO₂C₂H₅ requires | C, 67.8; | H.69; | N, 5.8; | Cl, 2.6% |

Water Assay Found: 3.74% w/w H₂O=1 mol H₂O

### EXAMPLE 31

### N-(4′-Acetyl[1,1′-biphenyl]-4-yl)-4-methoxy-3-[3-(methylamino)propyl]benzamide

A refluxing solution of the product obtained in Example 23 (408 mg) in dry 1,4-dioxan (25 ml), under nitrogen, was treated with manganese (IV) oxide (509 mg) and the mixture was heated at reflux for 1.75 h. The hot reaction mixture was filtered, washed with dichloromethane, and the filtrate evaporated to give an off-white solid (348 mg). The solid was purified by FCC eluting with System A (100:8:1) to give an off-white solid (234 mg). The solid was crystallised from isopropanol (30 ml) to give the title compound (81 mg) as an off-white solid m.p. 195-196° C.

T.l.c. System A (50:8:1) Rf 0.33.

### EXAMPLE 32

### N-[2′-chloro-4′-(hydroxymethyl)[1,1′-biphenyl]-4-yl]-3-[3-(dimethylamino)propyl]-4-methoxybenzamide

A hot solution of Intermediate 30 (1.07 g), and Intermediate 32 (0.81 g), in DME (50 ml), was treated with anhydrous sodium carbonate (0.63 g) in water (25 ml), followed by tetrakis(triphenylphosphine)palladium (0) (130 mg), and heated at reflux under nitrogen for 6 h. When cool, aqueous sodium bicarbonate was added (8%, 20 ml), and the reaction mixture was evaporated to dryness. The resultant solid was purified by FCC eluting with System A (93:7:0.7) to give the title compound (918 mg). This was re-purified by SPC eluting with the same eluent as before to give the title compound as a cream-coloured foam (703 mg) m.p. 68-70° C.

T.l.c. System A (93:7:0.7) Rf 0.18.

### EXAMPLE 33

### N-(2′-Chloro-4′-formyl[1,1′-biphenyl-4-yl)-3-[3-(dimethylamino)propyl]-4-methoxybenzamide hydrochloride

A solution of Example 32 (598 mg) in dry 1,4-dioxan (40 ml) was treated with activated manganese (IV) oxide (803 mg) and heated at reflux under nitrogen for 4 h. The reaction mixture was then filtered when cold, and the filter pad was washed with hot ethanol (1 litre). The filtrate was then evaporated in vacuo and the residue purified by SPC eluting with System A (95:5:0.5) to give the free base of the title compound as a gum (389 mg) which was dissolved in minimum ethyl acetate, and treated with ethereal HCl to pH1 to give the title compound as a cream solid (321 mg) m.p. 160-162° C.

| | | | | |
|---|---|---|---|---|
| Analysis Found: | C, 63.2; | H, 5.9; | N, 5.3. | Cl, 14.3 |
| C₂₆H₂₇CIN₂O₃.HCl 0.39 H₂O | C, 63.2; | H, 5.9; | N, 5.7; | Cl, 14.3% |
| requires | | | | |

Water Assay Found 1.43% H₂O w/w=0.39 mol H₂O.

### EXAMPLE 34

### 3-[3-(Dimethylamino)propyol-4-methoxy-N-[4′-(methoxymethyl)-2′-methyl [1,1′-biphenyl-yl]benzamide

Intermediate 30 (263 mg), Intermediate 34 (150 mg), tetrakis(triphenylphosphine)palladium (0) (50 mg), aqueous sodium carbonate (1N; 2 ml) and DME (8 ml) was refluxed under nitrogen for 4 h. The mixture was added to water (50 ml) and extracted with dichloromethane (3×50 ml). The dried extract was evaporated and the residue was purified by FCC eluting with System A (240:10:1) to give the title compound as a colourless gum (90 mg).

T.l.c. System A (90:10:1), Rf 0.6.

n.m.r. (250 MHz, CDCl₃) δ 1.82 (2H, m), 2.26 (6H, s), 2.3 (3H, s), 2.35 (2H, t), 2.7 (2H, t), 3.43 (3H, s), 3.89 (3H, s), 4.49 (2H, s), 6.92 (1H, d), 7.2-7.26 (3H, dd+d+d), 7.32 (2H, ½ AA′BB′), 7.67-7.81 (4H, dd+d+½ AA′BB′), 7.9 (1H, br.s).

### EXAMPLE 35

### 3-[3-(Dimethylamino)propyl]-N-(4′-hydroxy-2′-methyl[1,1′-biphenyl]-4-yl)-4-methoxybenzamide hydrochloride

Thionyl chloride (3 ml) was added to the hydrochloride salt of Intermediate 2 (0.634 g) and the mixture stirred at reflux for 5 min. When cool, the solution was evaporated, then co-evaporated with toluene (2×10 ml). A portion of the residue (0.534 g) was added portionwise over 40 min to a stirred mixture of Intermediate 37 in dry pyridine (6.6 ml) (ice-salt cooling) under nitrogen. The mixture was stirred at room temperature for 35 min, then at 90° C. for 90 min. Further dry pyridine (3 ml) was added during this time. When cool, aqueous sodium bicarbonate (8%, 20 ml) was added portionwise and the mixture evaporated. The residue was treated with refluxing ethanol (140 ml) and adsorbed onto silica gel (Merck 7734, 12 ml). The product was purified from a plug of this silica gel by SPC and eluting with System A (239:10:1 to 1857:130:13) to yield the free base of the title compound as a pale cream solid (0.581 g). A portion of this (0.400 g) was dissolved in the minimum quantity of refluxing ethanol and treated with ethereal hydrogen chloride to yield the title compound as a white powdery solid, (0.241 g).

T.l.c. System A (114:10:1) Rf 0.17.

| | | | | |
|---|---|---|---|---|
| Analysis Found: | C, 67.9; | H, 7.0; | N, 6.0; | Cl, 7.8 |
| C₂₆H₃₀N₂O₃.HCl.0.22H₂O | C, 68.0; | H, 6.9; | N, 6.1; | Cl, 7.8% |
| requires: | | | | |

Water assay Found 0.88% H₂O w/w=0.22 molH₂O.

Similarly prepared was:

### EXAMPLE 36

### N-[4′-[(Acetylaminomethyl][1,1′-biphenyl]-4-yl]-3-[3-(dimethylamino) propyl]-4-methoxybenzamide

as a cream-coloured solid (851 mg) m.p. 225-226° C.

| | | | |
|---|---|---|---|
| Analysis Found: | C, 72.8; | H, 7.2; | N, 9.0. |
| C₂₈H₃₃N₃O₃ 0.027 H₂O requires | C, 73.1; | H, 7.2; | N, 9.1%. |

Water Assay Found: 0.10% 10 w/w=0.027 mol H₂O

From the hydrochloride salt of Intermediate 2 (0.87 g) and Intermediate 40 (0.78 g). Purification by SPC eluting with System A (93:7:0.7) afforded the title compound.

### EXAMPLE 37

### 4′-[[3-[3-(Dimethylamino)propyl]4-methoxybenzoyl]amino]-2′-methyl[1,1′-biphenyl]-4-ol acetate(ester)citrate

Acetic anhydride (1.00 ml) was added at 0° under nitrogen to a stirred solution of the product of Example 48 (280 mg) in pyridine (10 ml), and the solution stored at 0° for three days. The solution was evaporated and the residue treated with aqueous saturated sodium bicarbonate (30 ml). The mixture was extracted with ethyl acetate (6×50 ml) and the combined, dried organic extracts were evaporated to give the crude free base (221 mg). A portion of the crude free base (195 mg) was treated with citric acid monohydrate (120 mg), followed by ethanol (3 ml) and stirred at reflux to effect solution. On cooling, the title compound crystallised as fine white crystals (74 mg).

T.l.c. System A (89:10:1), Rf 0.39.

| | | | |
|---|---|---|---|
| Analysis Found: | C, 60.7; | H, 6.2; | N, 4.1; |
| C₂₈H₃₂N₂O₄.C₆H₈O₇.1.02H₂O requires: | C, 60.8; | H, 6.3; | N, 4.2% |

Water assay Found: H₂O, 2.75% w/w=1.02 H₂O

### EXAMPLE 38

### N-(2′-Chloro-4′-methyl[1,1′-biphenyl]-4-yl)-3-[3-(dimethylamino)propyl]-4-methoxybenzamide

A hot solution of Intermediate 41 (250 mg) and Intermediate 20 (575 mg) was treated according to the method of Example 32. Purification by SPC eluting with System A (93:7:0.7), gave the title compound as a cream-coloured foam (283 mg) m.p. 63-66° C.

T.l.c. System A (85:15:1.5) Rf 0.49.

Similarly prepared were:

### EXAMPLE 39

### N-(4′-Acetyl-2-methyl[1,1′-biphenyl]-4-yl)-3-[3-(dimethylamino)propyl]-4-methoxybenzamide hydrochloride as a white solid (488 mg) m.p 264-266° C.

| | | | | |
|---|---|---|---|---|
| Analysis Found: | C, 69.2; | H, 6.8; | N, 5.6; | Cl, 7.6. |
| C₂₈H₃₂N₂O₃.HCl.0.06 H₂O | C, 69.7; | H, 6.9; | N, 5.8; | Cl, 7.35% |
| requires | | | | |

Assay Found: 0.25% w/w H₂O=0.06 mol H₂O.

From Intermediate 45 (700 mg) and Intermediate 46 (0.28 g). Purification by SPC eluting with System A (890:100:10) afforded the free base of the title compound (0.56 g) as an orange/yellow gum. A solution of this gum (530 mg) in ethyl acetate (10 ml) was treated with ethereal hydrogen chloride until the acidity was pH1. The solid was filtered off to yield the title compound.

### EXAMPLE 40

### 3-[3-(Dimethylamino)propyl]-N-(4′-formyl-2′-methyl[1,1′-biphenyl[-4-yl)-4-hydroxybenzamide

as a light yellow foam (0.31 g).

T.l.c. System G (923:70:7) Rf 0.32.

| | | | |
|---|---|---|---|
| Analysis Found: | C, 73.4; | H, 6.8; | N, 6.4. |
| C₂₆H₂₈N₂O₃.0.31 H₂O.0.05C₂H₅OH | C, 73.9; | H, 6.8; | N, 6.6%. |
| requires | | | |

Water Assay Found: 1.33% w/w H₂O=0.31 mol H₂O.

From Intermediate 21 (0.51 g) and Intermediate 28 (0.34 g). Purification by SPC eluting with a gradient of System G (967:30:3 to 956:40:4) gave a light yellow foam which on drying gave then title compound.

### EXAMPLE 41

### 3-[3-(Dimethylamino)propyl]-N-[4′-(hydroxymethyl)-2,2′-dimethyl[1,1′-biphenyl]-4-yl]-4-methoxybenzamide

as a cream-coloured foam (1.15 g) m.p. 68-70°

| | | | |
|---|---|---|---|
| Analysis Found: | C, 73.2; | H, 7.6; | N, 5.9. |
| C₂₈H₃₄N₂O₃.0.3H₂O.0.5 C₂H₅OH | C, 73.3; | H, 8.0; | N, 5.9%. |
| requires: | | | |

Water Assay Found: 1.19% H₂O w/w=0.3 mol H₂O

From Intermediate 47 (1.06 g) and Intermediate 45 (1.3 g). Purification by SPC eluting with System A (90:10:0.5) afforded the title compound.

### EXAMPLE 42

### 3-[3-(Dimethylamino)propyl]-N-(4′-formyl-2,2′-dimethyl[1,1′-biphenyl]-4-yl)-4-methoxybenzamide

A solution of the product of Example 41(1.19 g) in dry 1,4-dioxan (12 ml), was treated with activated manganese (IV) oxide (1.61 g), and heated at reflux under nitrogen for 3 h. When cool, the reaction mixture was filtered, and the filter cake was washed with hot ethanol (2.5 litres). The washings were combined and evaporated in vacuo to give a yellow solid, which was purified by SPC eluting with System A (96:4:0.4) to give the title compound as a yellow solid (0.91 g) m.p. 63-66° C.

| | | | |
|---|---|---|---|
| Analysis Found: | C, 74.4; | H, 7.1; | N, 6.0. |
| C₂₈H₃₂N₂O₃ 0.12 H₂O, 0.25 C₂H₅OH | C, 74.7; | H, 7.4; | N, 6.1%. |
| requires | | | |

Water Assay Found: 0.48% H₂O w/w=0.12 mol H₂O

### EXAMPLE 43

### N-(4′-Acetyl-2′-methyl[1,1′-biphenyl]-4yl)-3-[3-(dimethylamino)propyl]-4-methoxybenzamide

The hydrochloride salt of Intermediate 2 (0.15 g) was treated with thionyl chloride (4 ml) and stirred at reflux for 20 min. When cool, the solution was evaporated and then co-evaporated with toluene (2×20 ml). The residue was treated with Intermediate 49 (137 mg), followed by pyridine (5 ml) and then stirred at 110° under nitrogen for 18 h. When cool, aqueous saturated sodium bicarbonate (8%; 50 ml) was added and the mixture evaporated. The residue was purified by SPC eluting with System A (97:3:0.3) to give the title compound as a light brown oil. Re-evaporating the sample with ethyl acetate (50 ml) transformed the oil into a light brown solid (171 mg), m.p. 110-112° C.

| | | | |
|---|---|---|---|
| Analysis Found: | C, 74.9; | H, 7.5; | N, 5.9 |
| C₂₈H₃₂N₂O₃ 0.15 H₂O requires | C, 75.2; | H, 7.3; | N, 6.3% |

Water Analysis Found: 0.59% w/w H₂O=0.15 mol H₂O

### EXAMPLE 44

### 3-[3-(Dimethylamino)propyl]-N-[4′-(1-hydroxyethyl)-2′-methyl[1,1′-biphenyl]-4-yl]-4-methoxybenzamide

a solution of the product of Example 43 (200 mg) in ethanol (6 ml) was treated with sodium borohydride (34 mg) and stirred under nitrogen for 18 h at room temperature. Acetic acid (2 ml) was added, followed by aqueous saturated sodium bicarbonate (6 ml), and the mixture partially evaporated. The aqueous residue was extracted with ethyl acetate (3×25 ml), and the combined, dried organic extracts were evaporated. The residue was purified by SPC eluting with System A (97:3:0.3) to give the title compound as a cream coloured foam (140 mg).

T.l.c. System A (95:5:0.5) Rf 0.2

| | | | |
|---|---|---|---|
| Analysis Found: | C, 74.5; | H, 7.8; | N, 5.9. |
| C₂₈H₃₄N₂O₃ 0.2 CH₃CO₂C₂H₅ requires | C, 74.5; | H, 7.7; | N, 6.0% |

### EXAMPLE 45

### 3-[3-(Dimethylaminopropyl]-N-[4′-(1-hydroxymethyl)-2′-methyl[1,1′-biphenyl]-4-yl]-4-methoxybenzamide

A solution of the free base of the product of Example 3 (500 mg) in ethanol (25 ml) was added to a pre-reduced suspension of platinum on carbon (5%, 150 mg) and hydrogenated at room temperature and pressure until uptake ceased. The catalyst was filtered off and evaporated to yield a yellow liquid. The yellow oil was purified by FCC, eluting with System A (923:70:7) to give a cream-coloured foam (0.23 g). Further purification by SPC eluting with the same eluent afforded the title compound as a cream foam, (0.20 g) m.p 62-65° C.

| | | | |
|---|---|---|---|
| Analysis Found: | C, 74.35; | H, 7.5; | N, 6.0. |
| C₂₇H₃₂N₂O₃.0.34H₂O requires | C, 73.9; | H, 7.5; | N, 6.4%. |

Water Assay Found: 1.40% w/w H₂O=0.34mol H₂O.

### EXAMPLE 46

### 3-[3-(Dimethylamino)propyl]-N-(4′-formyl-2′-methyl[1,1′-biphenyl]-4-yl)-4-methoxybenzamide, hydrochloride (1:1)

2N Sodium bicarbonate solution (20 ml) was added to the ethanedioate (1:1) salt of the compound of Example 3 (656 mg). The resulting solution was extracted with ethyl acetate (4×20 ml), the extract dried and evaporated to yield the free base, (288 mg). Ethereal hydrochloric acid (5 ml) was added to the free base (288 mg), the solid filtered off, washed with ethyl acetate and dried under high vacuum at 60° to give the title compound as a cream-coloured solid (0.09 g), m.p. 112-115°.

| | | | |
|---|---|---|---|
| Analysis Found: | C, 68.3; | H, 6.6; | N, 5.7% |
| C₂₇H₃₀N₂O₃.HCl.0.40 H₂O requires | C, 68.4; | H, 6.8; | N, 5.9% |

Water assay Found 1.53% w/w H₂O =0.40mol H₂O.

### EXAMPLE 47

### 4′-[[3-[3-(Dimethylamino)propyl]-4-hydroxybenzoylamino]1,1′-biphenyl]-4-carboxamide

Sodium hydride (741 mg, 52% dispersion in oil,) was washed under nitrogen with hexane (3×10 ml), treated with dry DMF (8 ml) and then ethanethiol (1.18 ml, 0.99 g) in DMF (8 ml) was added dropwise over 5 min. After 1 h the free base of Example 10 (0.680 g) was added followed by DMF (8 ml). The solution was heated at 150° under nitrogen for 19 h. The cooled dark mixture was evaporated in vacuo, acidified with hydrochloric acid (5N) then basified to pH 8 by the addition of aqueous saturated sodium bicarbonate. The precipitate was filtered off, stirred with refluxing ethanol (1000 ml) and filtered to remove insoluble material. The filtrate was adsorbed onto silica gel (Art 9385, 5 g) and purified by FCC eluting with System A (37:7:0.7) to give a white powder (125 mg) which was recrystallised from hot ethanol (500 ml) to give the title compound as a white powder (60 mg)

T.l.c. System A (37:7:0.7) Rf 0.21.

| | | | |
|---|---|---|---|
| Analysis Found: | C, 71.2; | H, 6.7; | N, 9.8%. |
| C₂₅H₂₇N₃O₃0.17C₂HOH requires | C, 71.6; | H, 6.6; | N, 9.9%. |

### EXAMPLE 48

### 3-[3-(Dimethylamino)propyl]-N-(4′-hydroxy-2-methyl[1,1′-biphenyl]-4-yl)-4-methoxybenzamide

A catalytic quantity of tetrakis(triphenylphosphine)palladium (0) (143 mg) was added to a degassed refluxing mixture of Intermediate 45 (1.00 g), Intermediate 23 (684 mg) and sodium carbonate (287 mg) in DME (10 ml) and water (10 ml). The reaction mixture was heated at reflux for 6 h, under nitrogen. A further quantity of Intermediate 23 (340 mg) was added and the reaction mixture was heated at reflux for a further 6 h. The reaction mixture was added to aqueous sodium hydrogen carbonate (1M; 150 ml) and extracted with dichloromethane (5×50 ml). The combined extracts were dried, filtered and evaporated to give a brown gum (1.82 g). The gum was purified by FCC eluting with System A (100:8:1) to give a colourless gum(696 mg), which was dissolved in boiling ethyl acetate (75 ml) with a minimum of methanol. The hot solution was filtered, the filtrate was concentrated to (25 ml) and the solution allowed to cool to room temperature overnight. The resulting solid was filtered off, washed with ether an dried under vacuum to give the title compound as an off-white crystalline solid (445 mg) m.p. 184-186° C.

| | | | |
|---|---|---|---|
| Analysis Found: | C, 73.9; | H, 7.4; | N, 6.5. |
| C₂₆H₃₀N₂O₃.0.1H₂O requires | C, 74.3; | H, 7.2; | N, 6.7%. |

Water analysis indicates 0.1 H₂O

### EXAMPLE 49

### 3-[3-(Dimethylamino)propyl]-N-4′-formyl[1,1′-biphenyl]-4-yl)-4-methoxybenzamide maleate (1:1)

Activated manganese (IV) oxide (1.89 g) was added to a hot solution of the free base of Example 24 (1.67 g) in dry 1,4-dioxan (120 ml) and the mixture stirred at reflux for 2 h. The cooled suspension was filtered and evaporated. The product was purified by FCC eluting with System A (189:10:1) to give a cream-coloured yellow solid (1.20 g). A portion of this (0.30 g) was treated with maleic acid (0.13 g) and ethanol added in portions until a solution was obtained at reflux. On cooling the title compound crystallized as cream crystals (0.15 g), m.p. 173° C.

| | | | |
|---|---|---|---|
| Analysis Found: | C, 67.4; | H, 6.0; | N, 5.0. |
| C₂₆H₂₈N₂O₃.C₄H₄O₄ requires | C, 67.65; | H, 6.1; | N, 5.3. |

### EXAMPLE 50

### 3-[3-(Dimethylamino)propyl]-N-[4′-[(hydroxyimino)methyl][1,1′-biphenyl]-4-yl]-4-methoxybenzamide maleate (1:1)

Hydroxylammonium chloride (0.471 g) was added to the free base of Example 49 (0.399 g), the mixture treated with dry pyridine (7.85 ml) and stirred under nitrogen at room temperature for 18 h. Sodium bicarbonate solution (8%, 47 ml) was added and the mixture stirred under nitrogen for 10 min. The mixture was then evaporated, water (100 ml) was added and the aqueous phase extracted with dichloromethane then chloroform. The combined, dried organic extracts were evaporated to give a white fluffy solid (0.388 g). A portion of this (0.348 g) was treated with maleic acid (0.145 g) and methanol added until a solution was formed at reflux. On cooling, the solution gave the title compound as a pale yellow solid (0.226 g) m.p. 187-189° C.

| | | | |
|---|---|---|---|
| Analysis Found: | C, 65.5; | H, 6.4; | N, 7.6. |
| C₂₆H₂₉N₂O₃.C₄H₄O₄.0.165H₂O requires | C, 65.4; | H, 6.1; | N, 7.6%. |

Water Assay 0.54% H₂O=0.165 mol H₂O

### EXAMPLE 51

### (E)-3-[3-(Dimethylamino)propyl]-4-methoxy-N-[4′-[methoxyimino)methyl]-2′-methyl[1,1′-biphenyl]-4-yl]benzamide hydrochloride

A mixture of the free base of the product of Example 3 (557 mg) and O-methylhydroxylamine hydrochloride (756 mg) was treated with pyridine (8 ml) and stirred at 23° for 18 h. Aqueous saturated sodium bicarbonate (5 ml) was added and the mixture evaporated. The residue was treated with aqueous saturated sodium chloride (200 ml) and extracted with ethyl acetate (5×200 ml). The combined, dried organic extracts were evaporated, the residue was stirred in hot ethyl acetate (120 ml) for 15 min, then filtered and on cooling, the filtrate deposited the title compound as fine white crystals (175 mg).

T.l.c. System A (89:10:1) Rf 0.34.

| | | | | |
|---|---|---|---|---|
| Analysis Found | C, 67.15; | H, 7.0; | N, 8.1; | Cl, 7.2. |
| C₂₈H₃₃N₃O₃.HCl.0.2H₂O requires | C, 67.3; | H, 6.9; | N.8, 4; | Cl, 7.1% |

Water assay Found: H₂O, 0.73% w/w=0.2 mol

### EXAMPLE 52

### N-([1,1′-Biphenyl]-4-yl)-3-[3-(dimethylamino)propyl]-4-methoxybenzamide

A mixture of Intermediate 30 (600 mg), Intermediate 64 (400 mg), tetrakis (triphenylphosphine)palladium (0) (100 mg), aqueous sodium carbonate (4 ml) and DME (16 ml) was refluxed under nitrogen for 6 h. The mixture was added to water (50 ml) and extracted with dichloromethane (3×75 ml). The dried extract was evaporated and the residue was purified by FCC eluting with System F (190:10:1) to give the title compound as a white solid (115 mg).

T.l.c. System F (90:10:1), Rf 0.6

| | | | |
|---|---|---|---|
| Analysis Found | C, 74.8; | H, 7.0; | N, 6.9; |
| C₂₅H₂₈N₂O₂.0.6H₂O requires | C, 75.2; | H, 7.4; | N, 7.0% |

### EXAMPLE 53

### 4′-[[3-[3-(Dimethylamino)propyl]-4-methoxybenzoyl]amino]-3-methyl[1,1′-biphenyl]-4-methanol, acetate ester

A solution of the product of Example 45 (120 mg) in acetic anhydride (0.5 ml) was treated with pyridine (5 drops) and stirred at room temperature for 3 h. The solution was purified by FCC eluting with ethyl acetate:methanol:ammonia (90:10:1) to give the title compound as a colourless gum (10 mg).

T.l.c. ethyl acetate:methanol:ammonia (90:10:1), Rf 0.3

| | | | |
|---|---|---|---|
| Analysis Found | C, 71.3; | H, 7.3; | N, 5.7; |
| C₂₉H₃₄N₂O₄.0.8H₂O requires | C, 71.2; | H, 7.3; | N, 5.7% |

### EXAMPLE 54

### Methyl 4′-[[3-[3-(dimethylamino)propyl]-4-methoxybenzoyl]amino][1,1′-biphenyl]-4-carboxylate

A solution of Intermediate 51 (496 mg) in DMF (40 ml) was added to a mixture of pre-hydrogenated 5% palladium on carbon (0.25 g) in dry TEY (20 ml) and the stirred suspension hydrogenated at room temperature and pressure for 15 min. The mixture was filtered and the filtrate evaporated to give a cream solid (422 mg). A portion (406 mg) was crystallised from toluene (50 ml) to give a buff powder. The powder and the mother liquors were adsorbed from hot ethanol onto silica gel (Merck 9385) and purified by FCC eluting with System E (57:10:1) to give the title compound as a white solid (302 mg) m.p. 188-191.5°.

| | | | |
|---|---|---|---|
| Analysis Found: | C, 72.6; | H, 6.7; | N, 6.2 |
| C₂₇H₃₀N₂O₄ requires | C, 72.6; | H, 6.8; | N, 6.3% |

### EXAMPLE 55

### 2-Methoxyethyl 4′-[[3-[3-(dimethylamino)propyl]-4-methoxybenzoyl]amino]-2-methyl[1,1′-biphenyl]-4-carboxylate

Portions of 3-[3-(dimethylamino)propyl]-4-methoxybenzoyl chloride hydrochloride (623 mg) (prepared from the hydrochloride salt of Intermediate 2 and thionyl chloride) were added to a stirred solution of Intermediate 54 (649 mg) in pyridine (10 ml) at 0° C. under nitrogen over a period of 20 min. After 30 min at room temperature, the stirred solution was heated to 90° for a further 40 min. When cool, aqueous saturated sodium bicarbonate (20 ml) was added and the mixture evaporated. The residue was stirred in hot ethanol (30 ml) and the mixture adsorbed onto silica gel (Merck 7734, 15 ml). Purification by FCC eluting with System A (967:30:3 to 956:40:4) gave the title compound as a yellow liquid (0.79 g). The yellow liquid was stirred in hot ethanol and the mixture adsorbed onto silica gel (Merck 7734, 10 ml) and further purified by FCC eluting with System A (967:30:3) to give the title compound as a yellow oil (454 mg).

T.l.c. System A (956:40:4) Rf 0.19

| | | | |
|---|---|---|---|
| Analysis Found: | C, 70.3; | H, 7.1; | N, 5.65 |
| C₃₀H₂₇N₂O₅.0.31H₂O requires | C, 70.6; | H, 7.2; | N, 5.5% |

Water Assay Found: H₂O, 1.10% w/w=0.31 mol

### EXAMPLE 56

### 2-(Dimethylamino)ethyl 4′-[[3-[3-(dimethylamino)propyl]-4-methoxybenzoyl]amino]-2-methyl1,1′-biphenyl]-4-carboxylate

A stirred mixture of Intermediate 30 (0.8 g), Intermediate 55 (0.64 g), tetrakis(triphenylphosphine)palladium (0) (0.13 g) and anhydrous sodium carbonate (0.356 g) in water (25mil) and DME (50 ml) under nitrogen, was heated at reflux. After 18 h, the solution was cooled and the reaction contents adsorbed onto silica gel [Merck Art. 7734]. Purification by SPC eluting with System A (94:6:0.6) afforded the title compound as a pale yellow oil (0.352 g).

| | | | |
|---|---|---|---|
| Analysis Found | C, 70.7; | H, 7.8; | N, 7.6. |
| C₃₁H₃₉N₃O₄.0.28 H₂O requires | C, 71.2; | H, 7.6; | N, 8.0%. |

Water assay Found: 0.96% w/w H₂O=0.28 mol H₂O

n.m.r. (DMSO) δ 1.73 (2H,m), 2.19 (6H,s), 2.28 (8H, s & t), 2.4 (3H,s), 2.68 (4H, 2 xt), 3.92 (3H,s), 4.42 (2H,t), 7.15 (1H,brs), 7.38-7.46 (3H,m), 7.84-7.96 (6H,m), 10.2 (1H,brs).

### EXAMPLE 57

### 2,3-Dihydroxypropyl 4′-[[3-[3-(dimethylamino)propyl]-4-methoxybenzoyl]amino]-2-methyl[1,1′-biphenyl]-4-carboxylate

A stirred mixture of Intermediate 30 (0.743 g) and Intermediate 57 (0.603 g), tetrakis(triphenylphosphine)palladium (0) (0.120 g) and anhydrous sodium carbonate (0.440 g) in water (5 ml) and DME (5 ml) under nitrogen was heated at reflux. After 5 h, the solution was cooled and the reaction contents adsorbed onto silica gel [Merck Art 7734]. Purification by SPC eluting with System A (91:9:0.9) gave a white foam. The foam was re-columned, as before, to give slightly purer product (0.31 g). H.p.l.c. was performed on the foam to give pure title compound (0.210 g), m.p. 60-62° C. (decomp.)

| | | | |
|---|---|---|---|
| Analysis Found: | C, 67.6; | H, 7.0; | N, 5.1; |
| C₃₀H₃₆N₂O₆.0.34H₂O.0.45CH₃CH₂OH requires | C, 67.8; | H, 7.25; | N, 5.1% |

Water assay Found: 1.14% w/w H₂O=0.34 mol H₂O.

### EXAMPLE 58

### (±) 2-Methoxy-1-methylethyl 4′-[3-[3-(dimethylamino)propyl]-4-methoxybenzamido]-2-methyl[]1,1′-biphenyl]-4-carboxyate

A mixture of Intermediate 58 (322 mg) and Intermediate 30 (400 mg) in DME (10 ml) was treated with a solution of sodium carbonate (238 mg) in water (5 ml), followed by tetrakis (triphenylphosphine)palladium (0) (65 mg) and stirred at reflux under nitrogen for 5 h. The cooled mixture was evaporated, treated with water (30 ml) and extracted with ethyl acetate (5×50 ml). The combined, dried organic extracts were evaporated and the residue purified by SPC (Merck 7729) eluting with System A (967:30:3) to give the title compound as a pale brown glass (120 mg).

T.l.c. System A (945:50:5), Rf 0.16.

| | | | |
|---|---|---|---|
| Analysis Found | C, 71.0; | H, 7.3; | N, 5.1 |
| C₃₁H₃₈N₂O₅.0.49H₂O requires | C, 70.6; | H, 7.45; | N.5.3% |

Water assay found: 1.67% w/w H₂O=0.49 mol H₂O.

### EXAMPLE 59

### 3-[3-(Dimethylaminopropyl]-4-methoxy-N-[3-methyl-4-(3-oxa-1H-isobenzofuran-6-yl)phenyl]benzamide

A mixture of Intermediate 59 (220 mg), 5-bromo-1(3H)-isobenzofuranone (150 mg), palladium acetate (5 mg), tri(orthotolyl)phosphine (15 mg), triethylamine (1 ml) and DMF (2 ml) was refluxed (100°) under nitrogen for 4 h. The solution was added to water (25 ml) and extracted with dichloromethane (3×50 ml). The dried extract was evaporated and the residue was purified on a column of silica eluting with ethyl acetate:methanol:ammonia (190:10:1) to give the title compound as an orange foam (245 mg).

T.l.c. ethyl acetate:methanol:ammonia (190:10:1) Rf 0.2

| | | | |
|---|---|---|---|
| Analysis Found: | C, 70.5; | H, 6.5; | N, 5.7; |
| C₂₈H₃₀N₂O₄.H₂O requires | C, 70.6; | H, 6.7; | N, 5.9% |

### EXAMPLE 60

### 2-Methoxyethyl 4-[[3-[3-(dimethylamino)propyl]-4-methoxybenzoyl]amino]-3-methoxy-2-methyl[1,1′-biphenyl]-4-carboxylate

A mixture of Intermediate 59 (150 mg), Intermediate 60 (150 mg), palladium acetate (5 mg), tri(orthotolyl)phosphine (15 mg), triethylamine (1 ml) and DMF (2 ml) was heated at 100° for 5 h under nitrogen. The mixture was added to water (30 ml) and extracted with ethyl acetate (3×50 ml). The dried extract was evaporated and the residue was purified on a column of silica eluting with System F (190:10:1) to give the title compound as a yellow gum (94 mg).

T.l.c. System F (90:10:1) Rf 0.4

| | | | |
|---|---|---|---|
| Analysis Found: | C, 65.8; | H.7.3; | N, 5.0; |
| C₃₁H₃₈N₂O₆.2H₂.O requires | C, 65.3; | H, 7.4; | N, 4.9% |

### EXAMPLE 61

### N-[4-(3-Oxa-1H-isobenzofuran-6-yl)phenyl-3-[3-(dimethylamino)propyl]-4-methoxybenzamide

A mixture of Intermediate 30 (500 mg), 5-bromo-1(3H)-isobenzofuranone (320 mg), tetrakis(tiphenylphosphine)palladium (0) (100 mg), aqueous sodium carbonate (4 ml) and DME (15 ml) was refluxed under nitrogen for 2 h. The resulting mixture was treated with brine (50 ml) and extracted with dichloromethane (3×70 ml). The dried extract was evaporated and the residue was purified by chromatography eluting with System F (190:10:1) followed by (90:10:1) to give the title compound as a white solid (370 mg)

T.l.c. System F (190:10:1) Rf 0.4

| | | | |
|---|---|---|---|
| Analysis Found | C, 72.1; | H, 6.4; | N, 6.2 |
| C₂₇H₂₈N₂O₄.0.3H₂O requires | C, 72.1; | H, 6.2; | N.6.2% |

### EXAMPLE 62

### (±)-[4′-[[3-[3-(Dimethylamino)propyl]-4-methoxybenzoyl]amino]-3-methyl[1,1′-biphenyl]-4-yl]methyl 2-methoxypropanoate

DMF (1drop) was added to a solution of (±)-2-methoxypropanoic acid (52 mg) and oxalyl chloride (63 mg) in dichloromethane (2 ml). The solution was stirred until effervescence ceased (30 min) and was added dropwise to a solution of the product of Example 45 (200 mg) in dichloromethane (5 ml) under nitrogen. The resulting suspension was evaporated and the residue was triturated with ether to leave a solid. The solid in dichloromethane (5 ml) and triethylamine (2 ml) was treated dropwise with a solution of the acid chloride (prepared as above) in dichloromethane (2 ml) and stirred for 1 hour. The mixture was added to aqueous sodium carbonate (1M; 20 ml) and extracted with dichloromethane (3×50 ml). The dried extract was evaporated and the residue was purified by FCC eluting with System F (190:10:1) to give the title compound as a colourless gum (67 mg).

T.l.c. System F (90:10:1) Rf 0.6

n.m.r. δ 1.45 (3H,d), 1.80 (2H,m), 2.25 (6H,s), 2.30 (3H,s), 2.35(2H,m), 2.7(2H,m), 3.41 (3H,s), 3.9 (3H,s), 3.95 (1H,q), 5.2 (2H,s), 6.92 (1H,d), 7.22-7.26 (>3H,m), 7.32 (2H, ½ AA′BB′), 7.68-7.73 (3H,m), 7.77 (1H,dd), 7.87 (1H,br.s).

### EXAMPLE 63

### 4′-[[3-[1-(Dimethylamino)propyl]-4-methoxybenzoyl]amino]-2-methyl-N-(methylsulfonyl)[1,1′-biphenyl]-4-carboxamide

A mixture of Intermediate 30 (250 mg), Intermediate 61 (200 mg), palladium (II) acetate (5 mg), tri(orthotolyl)phosphine (15 mg), DMF (2 ml) and triethylamine (1 ml) was refluxed under nitrogen for 3 h. The cooled mixture was treated with water (30 ml) and extracted with dichloromethane (3×50 ml). The dried extract was evaporated and the residue was purified by FCC eluting with System F (90:10:1) to give a pale yellow solid. The solid was triturated with hot methanol (10 ml) to leave the title compound as a white powder (95 mg) m.p. 253-254°.

T.l.c. System F (90:10:1) Rf 0.2.

### EXAMPLE 64

### N-[4-(4H-1,3-Benzodioxin-7-yl)phenyl]-3-[3-(dimethylamino)propyl]-4-methoxybenzamide

A mixture of Intermediate 30 (150 mg), Intermediate 63 (100 mg), tetrakis(triphenylphosphine)palladium (0) (30 mg), aqueous sodium carbonate (2 ml) and DME (5 ml) was refluxed under nitrogen for ]h. The cooled mixture was treated with brine (50 ml) and extracted with dichloromethane (3×70 ml). The dried extract was evaporated and the residue was purified by FCC eluting with System F (190:10:1) to give the title compound as a white solid (62 mg).

T.l.c. System F (90:10:1) Rf 0.5

| | | | |
|---|---|---|---|
| Analysis Found: | C, 70.1; | H, 6.6; | N, 6.1; |
| C₂₇H₃₀N₂O₄.H₂O requires | C, 69.8; | H, 6.9; | N, 6.0% |

### EXAMPLE 65

### N-[4-(1,3-Benzodioxol-5-yl)phenyl]-3-[3-(dimethylamino)propyl]-4-methoxybenzamide

A mixture of Intermediate 30 (300 mg), 5-bromo-1,3-benzodioxole (200 mg), tetrakis(triphenylphosphine)palladium (0) (50 mg), aqueous sodium carbonate (2 ml) and DME (8 ml) was refluxed under nitrogen for 6 h. The mixture was added to water (30 ml) and extracted with dichloromethane (3×50 ml). The dried extract was evaporated to give a white solid which was recrystallised from isopropanol (20 ml) to give the title compound as a white powder (185 mg). m.p. 190-192°.

| | | | |
|---|---|---|---|
| Analysis Found | C, 70.0; | H, 6.2; | N, 5.8 |
| C₂₆H₂₈N₂O₄.0.7H₂O requires | C, 70.2; | H, 6.6; | N.6.3% |

### EXAMPLE 66

### 3-[3-(Dimethylamino)propyl]-4-methoxy-N-[2′-methyl-4-(1,3-oxathiolan-2-yl)[1,1′-biphenyl]-4-yl]benzamide

A mixture of the product of Example 46 (500 mg) and 2-mercaptoethanol (0.073 ml) in dichloromethane (20 ml), under nitrogen was cooled (0°) and treated wth boron trifluoride etherate (0.026 ml). Stirring was maintained at 0° for 1 h, then at 20° for 18 h. The mixture was then washed with 8% sodium bicarbonate (30 ml), and the aqueous layer was extracted with dichloromethane (20 ml). The combined dried extracts were evaporated to give a pale yellow foam which was purified by FCC eluting with System A (150:8:1) to give the title compound as a colourless foam (173 mg).

T.l.c. System A (100:8:1) Rf 0.47

| | | | |
|---|---|---|---|
| Assay Found: | C, 69.75; | H, 6.9; | N, 5.55; |
| C₂₉H₃₄N₂O₃S.0.45H₂O requires | C, 69.85; | H, 7, 05; | N, 5.6% |

Water Determination Found 1.56% w/w=0.45 mol % H₂O

Also isolated from the columns was the corresponding bis-(hydroxyethyl sulphide).

### EXAMPLE 67

### 3-[3-(Dimethylamino)propyl]-N-[4′-(1,3-dioxolan-2-yl)-2′-methyl[1,1′-biphenyl]-4-yl]-4-methoxybenzamide

The free base of Example 46 (460 mg) was dissolved in toluene (20 ml) and p-toluenesulphonic acid (224 mg) and ethylene glycol (0.12 ml) was added. The mixture was then heated to reflux in the presence of molecular sieves for 18 h. Further ethylene glycol (2 ml) was added, and heating maintained for a further 2 h. The cooled mixture was partitioned between 8% sodium bicarbonate solution (40 ml) and ethyl acetate (2×40 ml). The dried extracts were evaporated to give a pale yellow oil which was purified by FCC eluting with System A (200:8:1) to give the title compound as a colourless solid (254 mg).

T.l.c. System A (100:8:1) Rf 0.51.

| | | | |
|---|---|---|---|
| Assay Found | C, 72.3; | H, 7.3; | N, 5.8; |
| C₂₉H₃₄N₄O₂.0.43H₂O requires | C, 72.2; | H.7.3; | N, 5.8% |

Water Determination 1.63% w/w=0.43 mol H₂O

### EXAMPLE 68

### 3-[3-(Dimethylamino)propyl]-N-[4′-(1H-imidazol-1-ylmethyl)[1,1′-biphenyl]-4-yl]-4-methoxybenzamide

The hydrochloride salt of Intermediate 2 (0.154 g) and Intermediate 66 (0.14 g) was treated according to the method of Example 35. Purification by SPC eluting with System A (95:5:0.5) afforded the title compound as a cream-coloured solid (195 mg) m.p. 240-241° C.

T.l.c. System A (95:5:0.5) Rf 0.15.

### EXAMPLE 69

### [N-[3-(Dimethylamino)propyl]-4-methoxyphenyl]-2′-methyl-4-(5-methyl-1,2,4-oxadiazol-3-yl)[1,1′-biphenyl]-4-carboxamide

To a stirred solution of Intermediate 68 (141 mg) in dry pyridine (4 ml) was added thionyl chloride (0.04 ml). The mixture was stirred for 1 h at 20° and then a solution of Intermediate 27 (100 mg) in dry pyridine (2 ml) was added. The mixture was then stirred at 20° for 20 h. and 2N sodium carbonate (50 ml) added. The mixture was extracted with ethyl acetate (2×50 ml), the combined extracts dried and, after filtration, were evaporated to give an oil which was purified by FCC eluting with System A (100:8:1) to give the title compound as a beige solid (71 mg)

T.l.c. System A (100:8:1) Rf 0.28

| | | | |
|---|---|---|---|
| Assay Found: | C, 7.6; | H, 6.7; | N, 11.25; |
| C₂₉H₃₂N₄O₃ requires | C, 71.9; | H, 6.65; | N, 11.55% |

### Example 70

### 3-[3-(Dimethylamino)propyl]-4-methoxy-N-[2′-methyl-4′-(5-methyl-1,2,4-oxadiazol-3-yl)[1,1′-biphenyl]-4-yl]benzamide

A mixture of Intermediate 67 (322 mg) and Intermediate 30 (500 mg) in water (10 ml) and glyme (10 ml) containing sodium carbonate (444 mg) and tetrakis(triphenylphosphine)palladium (0) (30 mg) was heated to reflux under nitrogen for 1 h. The mixture was allowed to cool and silica gel (Merck 9385, 15 g) was added, and the solvents evaporated. The residue was chromatographed eluting with System A (200:8:1) to give the title compound as a colourless oil which crystallised on standing (363 mg) m.p. 163-165° C.

| | | | |
|---|---|---|---|
| Assay Found | C, 71.75; | H, 6.75; | N, 11.35; |
| C₂₉H₃₂N₄O₃ requires | C, 71.9; | H, 6.65; | N, 11.55% |

Similarly prepared was:

### EXAMPLE 71

### 3-[3-(Dimethylamino)propyl]-4-methoxy-N-[2′-methyl-4′-(3-methyl-1,2,4-oxadiazol-5-yl)[1,1′-biphenyl]-4-yl]benzamide

as a cream-coloured solid (159 mg) m.p. 166-167° C.

| | | | |
|---|---|---|---|
| Assay Found: | C, 71.75; | H, 6.75; | N, 11.3: |
| C₂₉H₃₂N₄O₃ requires | C, 71.9; | H, 6.65; | N, 11.55% |

From a mixture of Intermediate 30 (385 mg) and Intermediate 69 (248 mg) in water (10 ml) and glyme (10 ml) containing sodium carbonate (343 mg) and tetrakis(triphenylphosphine)palladium (0) (23 mg).

1. A compound of formula (I): 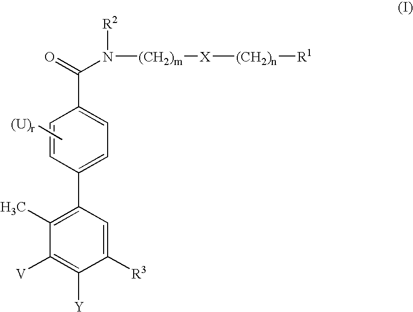
wherein
X is a bond or a phenyl group which may be optionally substituted;
R¹ is selected from an optionally substituted five- to seven-membered heterocyclic ring, an optionally substituted five- to seven-membered heteroaryl ring and an optionally substituted fused bicyclic ring;
R² is selected from hydrogen, C₁₋₆alkyl and —(CH₂)ₚ—C₃₋₇cycloalkyl;
or when X is a bond and m and n are both zero, R¹ and R², together with the nitrogen atom to which they are bound, form a five- to six-membered heterocyclic ring optionally containing one additional heteroatom selected from oxygen and nitrogen, which can be optionally substituted by C₁₋₄alkyl
R³ is the group 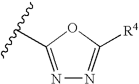
R⁴ is selected from hydrogen and C₁₋₄alkyl;
U is selected from methyl and halogen;
V and Y are each selected independently from hydrogen, methyl and halogen;
m and n are independently selected from 0, 1 and 2, wherein each carbon atom of the resulting carbon chain may be optionally substituted with up to two groups selected independently from C₁₋₆alkyl and the sum of m+n is from 0 to 4;
p is 0 or 1;
r is selected from 0, 1 and 2;
or a pharmaceutically acceptable salt or solvate thereof;
provided that the compound is not:
i) N-[4-methoxy-3-(4-methyl-1-piperazinyl)phenyl]-2′-methyl-5′-(5-methyl-1,3,4-oxadiazol-2-yl)-1,1′-biphenyl-4-carboxamide; or
ii) 2′-methyl-5′-(5-methyl-1,3,4-oxadiazol-2-yl)-N-[3-(4-methyl-1-piperazinyl)phenyl]-1,1′-biphenyl-4-carboxamide.

2. A compound according to claim 1 wherein R¹ is optionally substituted by up to three substituents selected from C₁₋₆alkyl, C₁₋₆alkoxy, oxy, halogen, hydroxyC₁₋₆alkyl, —N(C₁₋₆alkyl)₂, —CH₂—N(C₁₋₆alkyl)₂, —CO₂C₁₋₆alkyl, phenyl optionally substituted by halogen and benzyl optionally substituted by halogen and/or cyano.

3. A compound according to claim 1 wherein X is optionally substituted phenyl, and R¹ is selected from optionally substituted pyrrolidinyl, furyl, pyrrolyl, imidazolyl, pyrazolyl, tetrazolyl, oxazolyl, oxadiazolyl, piperidinyl, piperazinyl, morpholino, pyridyl, pyrimidinyl, thienyl, imidazolidinyl, benzimidazolyl and quinolyl; wherein the optional substituents for R¹ are selected independently from C₁₋₆alkyl, C₁₋₆alkoxy, oxy, halogen, hydroxyC₁₋₆alkyl, —N(C₁₋₆alkyl)₂ and —CH₂—N(C₁₋₆alkyl)₂.

4. A compound according to claim 1 wherein X is a bond, and R¹ is selected from an optionally substituted pyrrolidinyl, isoxazolyl, furyl, thienyl, imidazolyl, pyrazolyl, triazolyl, tetrazolyl, oxazolyl, thiazolyl, oxadiazolyl, piperidinyl, piperazinyl, morpholino, pyridyl, tetrahydrofuranyl, tetrahydrothiophenyl and quinolyl; wherein the optional substituents for R¹ are selected independently from C₁₋₆alkyl, C₁₋₆alkoxy, oxy, halogen, hydroxyC₁₋₆alkyl, —N(C₁₋₆alkyl)₂, —CH₂—N(C₁₋₆alkyl)₂, —CO₂C₁₋₆alkyl, phenyl optionally substituted by halogen and benzyl optionally substituted by halogen and/or cyano.

5. A compound according to claim 1 wherein R² is selected from hydrogen, C₁₋₄alkyl and —CH₂-cyclopropyl.

6. A compound according to claim 1 wherein R⁴ is C₁₋₄alkyl.

7. A compound according to claim 1 wherein m and n are independently selected from 0, 1 and 2, and the sum of m+n is from 0-3.

8. A compound according to claim 1 as defined in any one of Examples 1 to 44, or a pharmaceutically acceptable salt or solvate thereof.

9. A process for preparing a compound as claimed in claim 1 which comprises:
(a) reacting a compound of formula (XI) 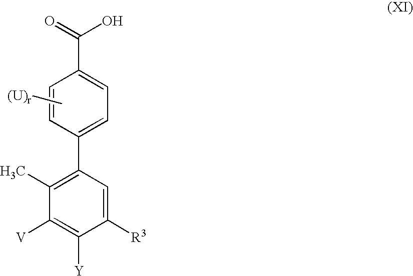
wherein R³, U, V, Y and r are as defined in claim 1, with a compound of formula (XII)
R¹(CH₂)ₙX(CH₂)ₘNR²H (XII)
wherein R¹, R², X, m and n are as defined in claim 1, under amide forming conditions;
b) reacting a compound of formula (XIII) 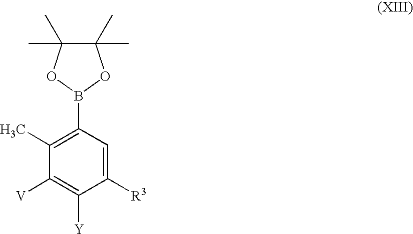
wherein R³, V and Y are as defined in claim 1, with a compound of formula (XIV) 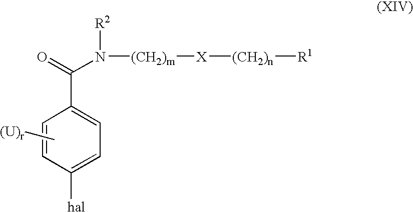
wherein R¹, R², U, X, m, n and r are as defined in claim 1 and hal is halogen, in the presence of a catalyst; or
c) reacting a compound of formula (XV) 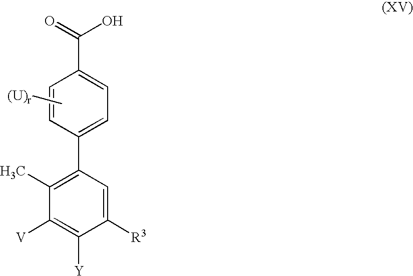
wherein R³, U, V, Y and r are as defined in claim 1, with a compound of formula (XVI)
R¹(CH₂)ₙX(CH₂)ₘNH₂ (XVI)
wherein R¹, X, m and n are as defined in claim 1, under amide forming conditions,
followed by reaction with a compound of formula (XVII)
R²-hal (XVII)
in which R² is as defined in claim 1 and hal is halogen, in the presence of a base.

10. A pharmaceutical composition comprising a compound according to claim 1 or a pharmaceutically acceptable salt or solvate thereof, in admixture with one or more pharmaceutically acceptable carriers, diluents or excipients.

11. A method for treating a condition or disease state mediated by p38 kinase activity or mediated by cytokines produced by the activity of p38 kinase comprising administering to a patient in need thereof a compound according to claim 1, but without provisos i) and ii), or a pharmaceutically acceptable salt or solvate thereof.

12-13. (Cancelled)
